(19) United States
(12) Patent Application Publication
(10) Pub. No.: US 2007/0011757 A1
(43) Pub. Date: Jan. 11, 2007

(54) Transgenic rodents as animal models for modulation of B1 bradykinin receptor protein

(76) Inventors: John W. Hess, Lansdale, PA (US); Robert J. Gould, Green Lane, PA (US); Douglas J. Pettibone, Chalfont, PA (US)

Correspondence Address: MERCK AND CO., INC, P O BOX 2000, RAHWAY, NJ, 07065-0907, US

(21) Appl. No.: 11/434,710

(22) Filed: May 16, 2006

Related U.S. Application Data
(63) Continuation of application No. 10/487,331, filed on Feb. 19, 2004, now abandoned.
(60) Provisional application No. 60/313,531, filed on Aug. 20, 2001.

(51) Int. Cl. A01K 67/027

(52) U.S. Cl. 800/14; 800/18

(57) ABSTRACT

Transgenic rats are generated which incorporate a primate B₁ bradykinin receptor transgene(s) into their genome. This B₁ bradykinin receptor gene is expressed in these transgenic rats, which results in binding of compounds which are selective for the primate form (such as the human form) of the receptor and not the rat form of the receptor. Therefore, the expressed transgenes within these transgenic lines mimic antagonist and agonist selectivity of the wild type primate B₁ bradykinin receptor. These transgenic animals are useful as a specific receptor occupancy model for modulators of the B₁ bradykinin receptor from the human or closely related species, as well as providing for an animal model system for assessment of the pharmacodynamic properties of such a B₁ bradykinin modulator(s).

### CROSS-REFERENCE TO RELATED APPLICATIONS

Not Applicable

### STATEMENT REGARDING FEDERALLY-SPONSORED R&D

Not Applicable

### REFERENCE TO MICROFICHE APPENDIX

Not Applicable

### FIELD OF THE INVENTION

The present invention relates to transgenic rodents which express a functional B₁ bradykinin receptor protein, preferably a mammalian B₁ bradykinin receptor protein and especially a functional non-human primate or human B₁ bradykinin receptor protein. The present invention is exemplified, but in no way limited by generation of transgenic rodents wherein random integration of a DNA sequence into the rodent genome has occurred, wherein the DNA sequence encodes the open reading frame of a human B₁ bradykinin receptor protein under control of a heterologous promoter. The present invention also relates to transgenic rodents which express functional modifications of a non-human primate or human B₁ bradykinin receptor protein, including but not limited to amino acid deletions, additions, substitutions, NH₂— or COOH-terminal truncations, splice variants, and the sort which provide for a protein with human B₁ bradykinin-like activity. The expressed transgenes within these transgenic lines mimic antagonist and agonist selectivity of the wild type B₁ bradykinin receptor. Therefore, the transgenic animals of the present invention are useful as a specific receptor occupancy model for modulators of a B₁ bradykinin receptor (such as a human B₁ bradykinin receptor), as well as providing for an animal model system for assessment of the pharmacodynamic properties of B, bradykinin modulators (e.g., human B₁ bradykinin modulators), such as antagonists or agonists of receptor activity.

### BACKGROUND OF THE INVENTION

Dray and Perkins (1993, TINS 16: 99-104) and Proud and Kaplan (1988, Annual Review Immunology 6: 49-83) define two mammalian bradykinin receptor subtypes, B₁ and B₂, based on their pharmacological properties. The nonapeptide bradykinin (BK) and the decapeptide Lys-BK (kallidin) are liberated from the large protein precursor kininogen by the proteolytic action of kallikreins. BK and kallidin both activate the B₂ receptor. These B₂ receptor agonists are then degraded by a carboxypeptidase to produce the B, receptor agonists des-Arg⁹BK and des-Arg¹⁰kallidin or by the angiotensin converting enzyme (ACE) to yield inactive peptides. BK and kallidin act as equipotent agonists at the B₂ bradykinin receptor subtype. In contrast, BK is totally inactive at the B₁ bradykinin receptor subtype. Des-Arg10,Leu9[Kallidin] (herein, “DALK”) is a peptide antagonist with structural similarities to kallidin.

The B₂ and B₁ bradykinin receptors are members of the superfamily of G-protein coupled receptors. Numerous mammalian B₁ and B₂ receptor genes have been isolated and characterized, including:

human B₁ bradykinin—U.S. Pat. Nos. 5,712,111 and 5,965,367, both issued to Menke et al. on Jan. 28, 1998 and Oct. 12, 1999, respectively, as well as Menke et al. (1994, J. Biol. Chem. 269:21583-21586).

rabbit B₁ bradykinin—MacNeil, et al., 1995, Biochem. Biophys. Acta 1264: 223-228.

mouse B₁ bradykinin—Hess et al., 1996, Immunopharmacology 33: 1-8;

rat B₂ bradykinin—McEachern, et al., 1991, Proc. Natl. Acad. Sci. 88, 7724-7728;

human B₂ bradykinin—Hess, et al. (1992, Biochem. Biophys. Res. Comm. 184: 260-268); and,

rat B₁ bradykinin—Jones, et al., 1999, Eur. J. Pharmacol. 374 (3), 423-433.

Hess et al. (1996, Immunopharmacology 33: 1-8) show that B₁ receptor agonist selectivity is species specific, namely when comparing the mouse, human and rabbit B₁ receptors.

Bock and Longmore (2000, Current Opin. in Chem. Biol. 4(4):401407) present a recent update of known modulators of B₁ and/or B₂ bradykinin receptor activity. As reviewed by the authors, it is widely held in the scientific community that B₂ receptors, but not B₁ receptors, are expressed in normal tissue. In contrast, biologic processes which result in inflammation, pain, tissue damage can rapidly induce B₁ receptor activity, as well as bacterial infection. The apparent inducibility of the B₁ receptor under such pathological conditions may provide a therapeutic window for the use of B₁ receptor antagonists as anti-inflammatory/analgesics, thus making the B₁ receptor an attractive drug target.

To this end, there remains a need for an animal model, including but not limited to a transgenic rat model, for use as a specific receptor occupancy model for modulators of the B₁ bradykinin receptor, as well as providing for an animal model system to assess pharmacodynamic properties of potential modulators for specificity to the human B₁ bradykinin receptor. The present invention meets this ongoing need by disclosing various transgenic rodent models which express a human B₁ bradykinin receptor protein.

### SUMMARY OF THE INVENTION

The present invention relates to non-human transgenic animal cells, non-human transgenic embryos, non-human transgenic animals (including but not limited to founder animals) and/or non-human transgenic littermates, where one or more transgene(s) encoding a functional form of a non-native mammalian B₁ bradykinin receptor has been stably integrated into the germ cells and/or somatic cells of the non-human animal. Preferred non-human transgenic cells are rodent cells and a preferred non-native B₁ bradykinin receptor gene for stable integration into the rodent genome is a primate B₁ bradykinin receptor gene.

In an exemplified embodiment of the present invention, these non-human transgenic animal cells and embryos are rat cells and embryos, which subsequently give rise to a transgenic rat, including initial founder animals, littermates, and subsequent animals which comprise members of the stable transgenic line which expresses a functional form of the human B₁ bradykinin receptor. These transgenic animals contain a genetic modification such that the modified animal now expresses a functional protein which has the pharmacological properties of the human B₁ bradykinin receptor, i.e. membranes prepared from the brain of the transgenic animal (exemplified herein with transgenic rats) have pharmacological properties that are distinct from the respective non-transgenic animal.

The present invention preferably relates to animal cells wherein at least one transgene encoding a functional form of a human B₁ bradykinin receptor has been stably integrated into the germ cells and/or somatic cells of the target animal. Additionally, the invention relates to non-human transgenic embryos, non-human transgenic founders, littermates and other transgenic animals which contain at least one transgene encoding a functional form of human B₁ bradykinin receptor. The transgenic animal cells, animals and littermates may express the non-native B₁ bradykinin receptor (e.g., a human B₁ bradykinin receptor) either in the presence or in the absence of the native (wild type) B₁ bradykinin receptor. In view of the methodology preferred for generating the transgenic rats of the present invention, a preferred transgenic cell, embryo and/or animal will contain alleles for both the native and transgenic, non-native B₁ bradykinin receptor.

The transgenic rat models as described herein will be useful to screen any potential modulator of receptor activity (e.g., antagonists or agonists), including but not necessarily limited to peptides, proteins, or non-proteinaceous organic or inorganic molecules. To this end, the present invention relates to processes for the production of the transgenic rats of the present invention and their offspring and their use for pharmacological testing. The invention further relates to methods of determining the selectivity and activity of potential modulators of the human B₁ bradykinin receptor by administering a test compound or compounds to the transgenic rat and measuring the effect of the compound on the activity of the human B₁ bradykinin receptor. To this end, the present invention relates to various occupancy assays with, for example, brain tissue, where the ability of a test compound to penetrate the blood brain barrier, distribute into the tissue and bind to the human B₁ receptor is measured.

As used herein, the term “functional” is used to describe a gene or protein that, when present in a cell or in vitro system, performs normally as if in a native or unaltered condition or environment. Therefore, a gene which is not functional (i.e., “non-functional”, “disrupted”, “altered”, or the like) will encode a protein which does not function as a wild type, native or non-altered protein, or encodes no protein at all. Such a non-functional gene may be the product of a homologous recombination event as described herein, where a non-functional gene is targeted specifically to the region of the target chromosome which contains a functional form of the gene, resulting in a “knock-out” of the wild type or native gene.

As used herein, a “modulator” is a compound that causes a change in the expression or activity of a mammalian B₂ or B₁ bradykinin receptor, such as a human B, bradykinin receptor, or causes a change in the effect of the interaction of the respective receptor with its ligand(s), or other protein(s), such as an antagonist or agonist.

As used herein in reference to transgenic animals of this invention, we refer to “transgenes” and “genes”. A gene is a nucleotide sequence that encodes a protein, or structural RNA. The gene and/or transgene may also include genetic regulatory elements and/or structural elements known in the art. As used and exemplified herein, a transgene is a genetic construct including a gene. The transgene is integrated into one or more chromosomes in the cells in an animal by methods known in the art. Once integrated, the transgene is carried in at least one place in the genome, preferably a chromosome, of a transgenic animal. The transgene of interest is incorporated into the target genome of the rat or other mammal, thus being introduced into their germ cells and/or somatic cells such that it is stably incorporated and is capable of carrying out a desired function. The transgene may also contain heterologous genetic regulatory elements and/or structural elements known in the art, such a heterologous promoter sequence and/or a heterologous enhancer sequence, which effects transcription of the open reading frame of the transgene within the target cell/animal. Such heterologous regulatory sequences are ‘fused’ or ‘operatively linked’ to the coding region so as to appropriately effect such gene expression. While a chromosome is the preferred target for stable incorporation of a transgene into the target animal, the term “genome” refers to the entire DNA complement of an organism, including nuclear DNA (chromosomal or extrachromosomal DNA) as well as mitochondrial DNA, which is localized within the cytoplasm of the cell. Thus, as noted previously, the transgenic rats of the present invention will stably incorporate one or more transgenes in either/or of the rat's germ cells or somatic cells (preferably both), such that the expression of the transgene (e.g., a functional form of a human B1 bradykinin gene) achieves the desired effect of presenting a specific receptor occupancy model for modulators of the human B1 bradykinin receptor as well as providing for an pharmacodynamic animal model system to study the selectivity of test compounds to modulate the human B1 bradykinin receptor. It is preferable to introduce the transgene into a germ line cell, thereby conferring the ability to transfer the information to offspring. If offspring in fact possess some or all of the genetic information, then they, too, are transgenic animals.

As used herein, the term “animal” may include all mammals, except that when referring to transgenic animals, the use of this term excludes humans. It also includes an individual animal in all stages of development, including embryonic and fetal stages. A “transgenic animal” is an animal containing one or more cells bearing genetic information received, directly or indirectly, by deliberate genetic manipulation at a subcellular level, such as by microinjection, targeted gene delivery such as by homologous recombination, or infection with recombinant virus. As noted above, this introduced DNA molecule (i.e., transgene) can be integrated within a chromosome, or it can be extra-chromosomally replicating DNA.

As used herein, “rodent” relates to a species which is a member of the order Rodentia, having a single pair of upper and lower incisors for gnawing, wherein the teeth grow continuously and a gap is evident between the incisors and grinding molars. Preferred examples include for generation of transgenic animals include, but are not limited to, Rattus norvegicus, Rattus rattus, and Mus musculus.

As used herein, “rat” relates to animals which from the point of systemic zoology belong to the genus Rattus. The transgenic animals of the present invention may be generated from any species of the genus Rattus, including but not limited to Rattus norvegicus and Rattus rattus.

As used herein, “founder” refers to a transgenic animal which develops from the microinjected egg or target cell, such as an embryonic stem cell that has been targeted by a homologous recombination event to, for example, replace a rodent gene with its human homologue. The founders are tested for expression of a functional gene by any suitable assay of the gene product.

As used herein, the term “line” refers to animals that are direct descendants of one founder and bearing one transgene locus stably integrated into their germline.

As used herein, the term “inbred line” refers to animals which are genetically identical at all endogenous loci. As used in the art, inbred lines may be used for including reproducibility from one animal to the next, ability to transfer cells or tissue among animals, and the ability to carry out defined genetic studies to identify the role of endogenous genes. Such inbred lines may be developed from such lines wherein the rats that are used for microinjection are members of established inbred strains.

As used herein, the term “genotype” is the genetic constitution of an organism.

As used herein, the term “phenotype” is a collection of morphological, physiological and/or biochemical traits possessed by a cell or organism that results from the interaction of the genotype and the environment. Included in this definition of phenotype is a biochemical trait wherein a non-native transgene has been introduced into the animal, thus altering its the genotypic profile, and whereby expression of this transgene(s) within the animal results in a new pharmacological selectivity to one or more chemical compounds, such a selectivity based on functional expression of the transgene(s) of interest. To this end, the term “phenotypic expression” relates to the expression of a transgene or transgenes which results in the production of a product, e.g., a polypeptide or protein, or alters the expression of the zygote's or the organism's natural phenotype.

As used herein, the terms “rat enolase promoter”, “rat neuron specific enolase promoter”, “NSE” and the such, are used interchangeably throughout this specification to refer to the promoter fragment used to exemplify the present invention, as discussed herein.

### BRIEF DESCRIPTION OF THE DRAWINGS

FIG. 1 shows the transgenic plasmid targeting construct, ratEnolase intron A hB1 polyA2 [rat neuron specific enolase promoter_CMV introna_human B1 bradykinin receptor coding sequence_BGH poly A signal CMV=human cytomegalovirus, BGH=bovine growth hormone (Construct #1; also referred to as NSE hB1)].

FIG. 2A-B shows the nucleotide sequence of the integrated transgene, ratEnolase intron A hB1 polyA2, as shown pictorially in FIG. 1A (SEQ ID NO:1).

FIG. 3A-B shows the structure (FIG. 3A) of the transcript generated from the ratEnolase intron A hB1 polyA2 targeting vector, also referred to as the NSE hB1 transcript, with the nucleotides sequence (FIG. 3B) disclosed as SEQ ID NO:2.

FIG. 4 shows the structure of the transgenic plasmid targeting construct, CMV promoter_CMV intron A_human B1 cds_BGH.

FIG. 5 shows a portion of the rat genomic plasmid targeting construct, CMV promoter_CMV Intron A_hB1 cds_IRES2/LacZ_BGH poly A (also referred to as pCMV B1 IZ).

FIG. 6A-B shows the nucleotide sequence of the integrated transgene CMV promoter_CMV Intron A_hB1 cds_IRES2/LacZ_BGH poly A (SEQ ID NO:3).

FIG. 7 shows a portion of the rat genomic plasmid targeting construct, Thy-1_hB1 cds-IZ pBS (also referred to as Thy1 hB1IZ).

FIG. 8A-C shows the nucleotide sequence of the integrated transgene Thy-1_hB1 cds-IZ pBS (SEQ ID NO:4).

FIG. 9A-C shows results from a saturation binding assay of ³H-DALK ([des-Arg¹⁰, Leu⁹]-Kallidin) to membranes isolated from transgenic rat brain tissue from (A): line 0004 (rat #1810); (B): line 0014 (rat #1813); and (C): line 0015 (rat #1814).

FIG. 10 shows autoradiograms of brain and spinal cord sections from NSE_hB1 line 0004 transgenic rats. Non-specific binding was determined with 0.3 nM [H-3] DALK in the presence of 200 nM of a non-peptide antagonist of the human B1 bradykinin receptor that has sub-nM affinity for the human B1 receptor. Total binding was determined using 0.3 nM [H-3] DALK. Regions of the brain and spinal cord that exhibit high levels of binding are indicated. Specific [H-3] DALK binding (total binding—nonspecific binding) is indicative of the level of human B1 bradykinin receptor expression. There is no detectable specific binding of [H-3] DALK in non-transgenic control rats.

### DETAILED DESCRIPTION OF THE INVENTION

The present invention relates to animal cells wherein at least one transgene encoding a functional form of non-native mammalian B₁ bradykinin receptor has been stably integrated into the germ cells and/or somatic cells of the target animal. To this end, the present invention relates to non-human transgenic embryos, non-human transgenic animals and non-human transgenic littermates which contain at least one transgene encoding a functional form of a non-native, mammalian B₁ bradykinin receptor. Preferred non-human transgenic cells are rodent cells and a preferred non-native B₁ bradykinin receptor gene for stable intergration into the rodent genome is a primate B₁ bradykinin receptor gene. Example of various non-human primate sources for isolated DNA molecules encoding B1 bradykinin receptor include but are not limited to members of the old world monkey group, such as various members of the genus Macaca, which included Macaca mulatta, the rhesus monkey; members of the new world monkeys such as members of the genus Sanuinus, which includes the tamarins; prosimians, which include Lemur members, and the great apes, such as the chimpanzee (Pan troglodytes), orangutan (Pongo pygmaeus) and gorilla (Gorilla gorilla).

Therefore, the present invention relates to non-human transgenic animal cells, non-human transgenic embryos, non-human transgenic animals and/or non-human transgenic littermates, where one or more transgene(s) encoding a functional form of a non-native mammalian B₁ bradykinin receptor has been stably integrated into the germ cells and/or somatic cells of the non-human animal. A preferred embodiment of this portion of the invention relates to transgenic rats which express a functional form of human B₁ bradykinin receptor, thus comprising rat transgenic cells and embryos, which subsequently give rise to a transgenic rats, including initial founder animals, littermates, and subsequent transgenic rats which represent a stable transgenic line expressing a functional form of the human B₁ bradykinin receptor. The transgene of interest contains a primate B₁ bradykinin receptor expression cassette operatively linked to regulatory sequences such as an enhancer and/or promoter fragment which are functional in the host animal, as well as a termination signal downstream of the B, open reading frame. The transgenic animal of the present invention allows for the investigation of pharmacological-based activity of an expressed transgene (e.g., human B₁ bradykinin receptor) in these animals, allowing for testing of the effect of certain test compounds within these transgenic animals and thus to perform preliminary tests for the development of new pharmaceutically active substances. It is evident from the data presented herein that non-human transgenic animals which incorporate a functional human B₁ bradykinin gene(s), or biologically equivalent form thereof, show a definable phenotype wherein the transgenic animal expresses an effective amount of the functional transgene product such that the transgenic animal now confers the selective pharmacological properties of the human B₁ bradykinin receptor. This phenotype is detailed herein via binding assays with membranes prepared from the brain of the transgenic rats, which are shown to have pharmacological properties that are distinct from the non-transgenic rats. This is evident in the binding data which demonstrates that human specific B₁ compounds, that have poor affinity for the rat B₁ receptor, have high affinity for membranes prepared from the brains of transgenic rats disclosed in the Example section. The endogenous rat receptor is unlikely to mask any phenotype. The radioligand ³H-DALK, has greater affinity for the human B₁ receptor than the rat B₁ receptor. This explains, in part, why no endogenous B₁ receptor activity is seen in non-transgenic rats using this ligand either by a whole brain homogenate assay or by receptor autoradiography of brain slices. In contrast, there is a binding site that is readily detectable using ³H-DALK in the exemplified transgenic rats expressing the human B₁ receptor using either of these techniques.

The transgenic rat models as described herein will be useful to screen any potential modulator of receptor activity (e.g., antagonists or agonists), including but not necessarily limited to peptides, proteins, or non-proteinaceous organic or inorganic molecules. The transgenic animals of the present invention provide for improved models to study the in vivo effects of test compounds on human B₁ bradykinin receptor activity. Previous to this disclosure, treatments of test animal with an agent to increase wild type B₁ bradykinin expression (such as bacterial lipopolysaccharides) gave varying results to the extent in which B₁ bradykinin expression was increased and which altered the properties of the blood-brain barrier. Even if successful, the properties of compounds selective for humans could not be assessed. To circumvent this problem, attempts were made to identify a species in which the pharmacological properties of the respective species matched the human B₁ bradykinin receptor; a disadvantage being that one species may have similar properties to the human with respect to one, but not all chemical series under consideration. Alternatively, species that are closely related genetically to human (such as non-human-primates) can be used. However, this alternative suffers from the low throughput in assaying compounds through such a non-transgenic model, such as a non-human primate.

One such assay, provided only as an example and not as a limitation, is the use of the transgenic animals of the present invention in an occupancy assay in the brain to assess the ability of test compounds to penetrate the blood brain barrier as well as the ability to distribute into the tissue and bind to the receptor. A type of occupancy receptor assay may be performed using the transgenic animal of the present invention and measuring the displacement of a known radiolabeled compound which binds to the human B₁ bradykinin receptor. For example, male transgenic or non-transgenic Sprague Dawley rats may be dosed orally with test compound and fasted over night. On the day of the experiment body weights are obtained. Administration of 50% PEG/D5W vehicle (iv) or 1% methocell (po) is used to determine total binding. For iv dosed compounds, rats are placed in a perspex rat restrainer for tail vein injection of vehicle, either a test compound and/or a second compound used for determination of non-specific binding. Seven and one-half minutes later, rats are returned to the restrainer and injected with 200 μCi/kg [³_] -test compound iv. For po dosed compounds, rats are dosed by gavage with vehicle or compound. Non-specific binding is determined. Sixty minutes later, rats are placed in the restrainer and injected with 200 μCi/kg [³ H]-test compound iv. Tail vein injections are through a 25 gauge, 1 inch needle on a 1 cc syringe. Tail veins are dilated by keeping rats warm and by wiping tails with an alcohol swab. Seven and ½ min after injection of isotope, animals are euthanized via CO₂ and the skull opened working from the base of the skull at the spinal cord opening forward to the orbital sockets. A slice of cortex approximately 100-150 mg is cut and immediately placed in a pre-tared polypropylene tube and weighed. Cold HEPES buffer (10 mM) [7.4 gms NaCl (150 mM), 4.8 gm HEPES (10 mM), 0.750 gm KCl (5 mM) per 2 liters of deionized water. pH brought to 7.4 with 1N NaOH. (NaCl & KCl: Fisher Scientific; HEPES (C₈H₁₈N₂O₄S: Boehringer Mannheim)] is added in 39 vols to each tube. Brains are homogenized using a polytron homogenizer at full speed for 10 sec. Five hundred μl of homogenate are immediately filtered in duplicate through a 25mm Pall A/E filter (pre-soaked in 0.2% PEI) using a filter unit (Hoeffer). Homogenate is pipetted onto filters with valve closed so homogenate covers entire surface area of filter, valve is then opened to allow filtering and washing), followed immediately by 5×5 ml washes of cold HEPES (5mM KCl, 150 mM NaCl, 10 mM HEPES) buffer. Each filter is placed in a scintillation vial and Ultima Gold scintillation fluid (10 ml) is added to each vial. Duplicate 500 μl aliquots of homogenate are pipetted into scintillation vials, 10 ml of Ultima Gold is added, and counted to measure total brain labelling. Samples are allowed to sit for 4 hours before counting with a tritium counting program. Isotope solution is counted with the tritium program to determine actual mCi concentration. A 0.001 ml sample is pipetted into a vial containing 10 ml of scintillation fluid (pipet tip is carefully wiped with a kimwipe). Calculations are as follows: (1) Percent accumulation of label (% Acc) for each sample=(Filter dpm/Homogenate dpm)×100; (2) Percent specific accumulation (% Sp Acc) for the entire assay=Mean % Acc for Total Binding−Mean % Acc for Non-specific binding; (3) Percent inhibition of binding (% Inh)=((Mean % Acc for Total Binding−% Acc for the sample))/% Sp Acc)×100; (4)1μCi=2.2×10⁶ DPM (0.001 ml isotope solution(0.2 mCi/ml)=4.4×10⁵ DPM). Dose of the test compound may be 200 μCi/kg BW for dosing volume of 0.15 ml in 150 gm rat (0.2 mCi/ml): 1 ml of 1 mC/ml (NEN)+4 ml saline.

A preferred embodiment of the present invention is the generation of transgenic rodents in which one transgene encoding a functional form of a human B₁ bradykinin receptor has been stably integrated into the germ cells and/or somatic cells of the target animal. However, the present invention relates to the generation of other transgenic, non-human animals, other than the preferred targets of rats and mice, which exhibit substantially similar phenotypic traits as the exemplified transgenic rats disclosed herein, including but not limited to cows, pigs, rabbits, guinea pigs, sheep, hamsters, and goats.

Also, the present invention preferably relates to animal cells wherein at least one transgene encoding a functional form of a human B₁ bradykinin receptor has been stably integrated into the germ cells and/or somatic cells of the target animal. Additionally, the invention relates to non-human transgenic embryos, non-human transgenic animals and non-human transgenic littermates which contain at least one transgene encoding a functional form of human B₁ bradykinin receptor. The transgenic animal cells, animals and littermates may express the non-native B₁ bradykinin receptor (e.g., a human B₁ bradykinin receptor) either in the presence or in the absence of the native (wild type) B₁ bradykinin receptor. In view of the methodology preferred for generating the transgenic rats of the present invention, a preferred transgenic cell, embryo and/or animal will contain alleles for both the native and transgenic, non-native B₁ bradykinin receptor. Typically, the transgene of interest contains a human B₁ bradykinin receptor expression cassette linked to regulatory sequences such as an enhancer and/or promoter fragment which are functional in the host animal, as well as a termination signal downstream of the B₁ open reading frame. In the preferred mode of generating a transgenic rat encoding human B₁ bradykinin receptor, the transgene is typically integrated into a host chromosomal location by nonhomologous integration. These transgenes may further comprise a selectable marker, such as a neo or gpt gene operably linked to a constitutive promoter, such as a phosphoglycerate kinase (pgk) promoter or HSV tk gene promoter linked to an enhancer (e.g., SV40 enhancer).

In some embodiments, such as the targeted insertion of the human B1 bradykinin gene into mice, the endogenous nonhuman B₁ alleles are functionally disrupted so that expression of endogenously encoded murine B₁ is suppressed or eliminated, so as to not interfere with expression of the human B₁ transgene. Transgenes may be incorporated into embryonic, fetal or adult pluripotent stem cells (Capecchi, 1991, Science 244: 1288-1292, see also U.S. Pat. Nos. 5,464,764; 5,487,992; 5,627,059; 5,631,153 and 6,204,061 issued Mar. 20, 2001) hereby incorporated by reference. Embryonic stem cells can be isolated from blastocysts cultivated in vitro and stably cultured within differentiation. For example, a transgene may be contained within a gene targeting vector, wherein the vector contains homologous arms (see Cappecchi, supra) which can be used to direct a transgene to a specific genomic site within the target ES cell. Such foreign DNA can be incorporated into the embryonic stem cells by electroporation. Embryonic stem cells which carry the transgene in the appropriate fashion are injected into the inner cell mass of blastocysts. A chimeric animal is generated which is then crossbred to obtain animals wherein all cell carry the transgene. Along with microinjection described below, ES cell-based techniques are a preferable methodology for generating transgenic mice. A common scheme to disrupt gene function by gene targeting in ES cells is to generate a targeting construct which is designed to undergo a homologous recombination with its chromosomal counterpart in the ES cell genome. The targeting constructs are typically arranged so that they insert additional sequences, such as a positive selection marker, into coding elements of the target gene, thereby functionally disrupting it. To this end, the present invention also relates to methods of producing nonhuman animals (e.g., non-primate mammals) that have the endogenous B₁ gene inactivated by gene targeting with a homologous recombination targeting construct. General principles regarding the construction of targeting constructs and selection methods are reviewed in Bradley et al., 1992, Bio/Technology 10: 534, which is hereby incorporated herein by reference.

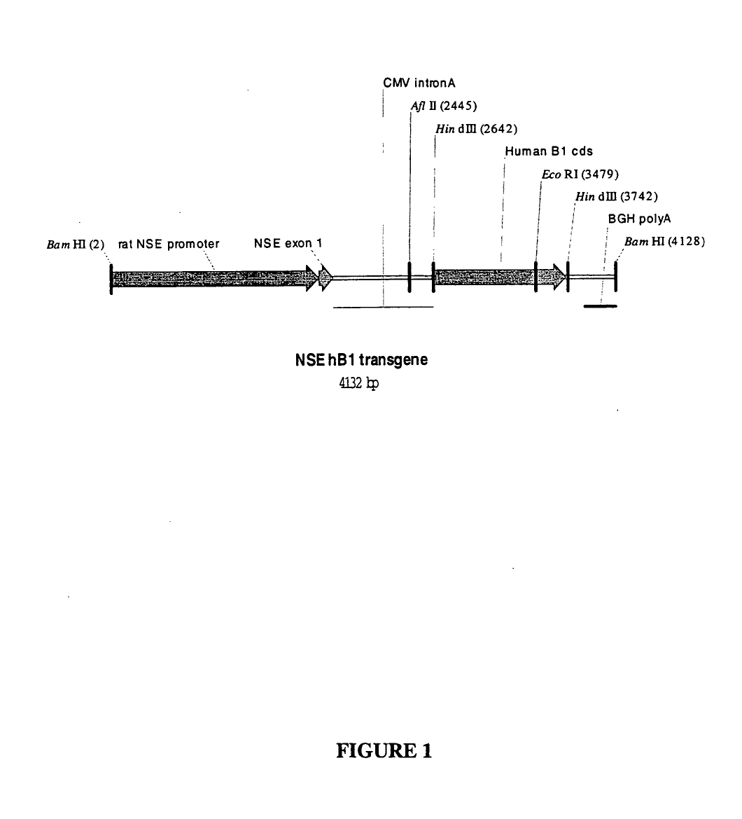

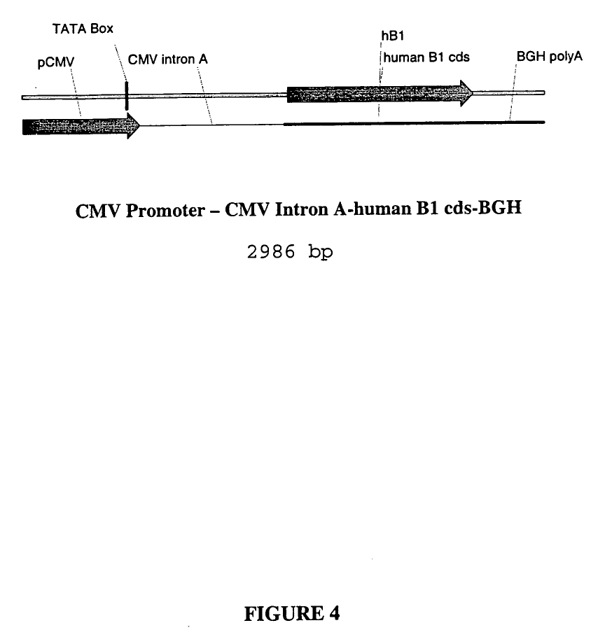

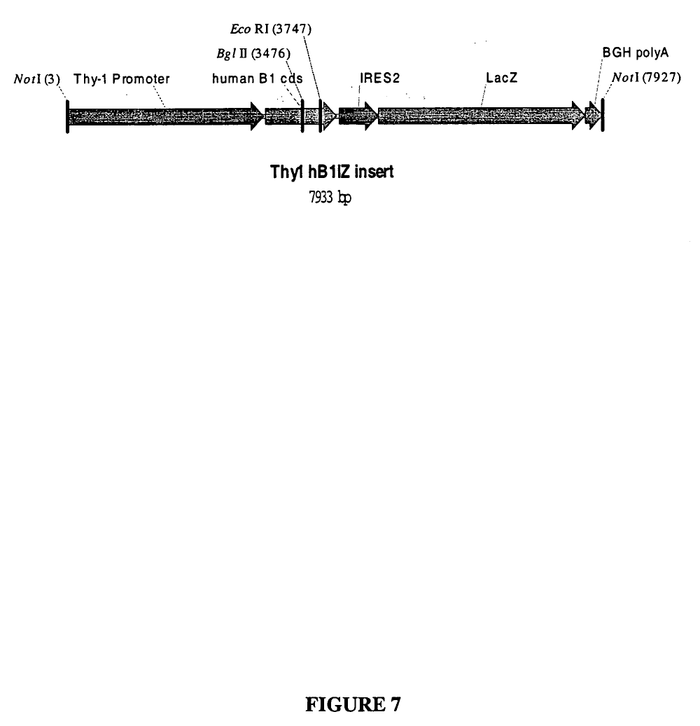

It is within the scope of the present invention to present a transgene encoding a mammalian form of interest to the host target cell as a DNA construction such that expression of the respective B₁ bradykinin receptor is controlled by various homologous or heterologous regulatory sequences operatively linked to the B₁ bradykinin gene. To present as examples, but certainly not as a limitation, see FIG. 1 (rat neuron specific enolase [NSE] promoter and CMV Intron A fused (i.e., operatively linked) to the human B₁ bradykinin receptor gene, which is upstream of the bovine growth hormone (BGH) transcriptional termination and polyadenylation signal). Animals which integrate this construct will present neuron specific expression within the central nervous system. In contrast, peripheral expression of the human B₁ bradykinin gene will occur via the integration construct shown in FIG. 4 (CMV promoter/Intron A fused to the human B₁ bradykinin receptor gene, which is upstream of a second open reading frame (LacZ) which is separated by an internal ribosome entry site (IRES), with a BGH termination signal downstream of the LacZ ORF). Also, the transgene construct shown in FIG. 7 (Thy-1 promoter fused to the human B₁ bradykinin receptor gene, which is upstream of a second open reading frame (LacZ) which is in turn separated by an internal ribosome entry site (IRES), with a BGH termination signal downstream of the LacZ ORF) should promote brain specific expression. These various constructions show that any of a myriad of promoter/transgene constructions may be generated for transfer into target cells for stable, genomic DNA integration and subsequent manipulation to produce a requisite transgenic animal. The exemplified constructs described herein also provide for the integration of discistronic constructs into a non-human transgenic animal. A preferred discistronic construct utilizes an internal ribosome entry site (IRES) to separate the respective open reading frames (ORF). It is preferred that a first ORF encode for a functional form of a primate B I bradykinin receptor while a second ORF encode a reporter gene which allows for easy detection of tissue and/or cellular specific expression. Various reporter genes are well known in the art and include, as an example but certainly not a limitation, LacZ, green fluorescent protein (GFP), chloramphenical acetyl transferase (CAT), alkaline phosphatase and luciferase.

A preferred method of generating a transgenic rat generally comprises first introducing DNA which includes the selected transgene into germ cells of the rat (typically fertilized eggs). These fertilized germ cells are then used to generate a complete, transgenic animal. The DNA is preferably introduced into the germ cells by known microinjection techniques, which comprises introducing the DNA into a germ cell through the aid of a microscope and a microinjector pipette which deposits intact DNA into one of the two pronuclei. Transgenic animals are selected which have incorporated into their genome at least one, and possibly more than one, selected transgene(s). At least one founder transgenic rat is selected for breeding so as to establish at least one transgenic rat line which contains the stably integrated transgene. This methodology is disclosed in U.S. Pat. No. 4,873,191. Other known techniques available in the art may be utilized to generate the transgenic animals of the present invention, including but not limited to in vitro fertilization using sperm as a carrier of exogenous DNA, electroporation or alternatively, transfection into a rat embryonic stem cell line may be utilized to directly target the transgene into the rat genome, followed by selection and introduction of selected, recombinant ES cells into a rat blastocyst. Various methodolodgy is reviewed in Mullins, et al., in Transgenic Animal: Generation and Use, Ch. 2.-Transgenic Rats, pp.7-9, Harwood Academic Publisher, 1997. Therefore, a preferred and well known method for preparing transgenic rats of the present invention includes the following steps: subjecting a female to hormonal conditions to promote superovulation (with a continuous infusion of a follicle stimulating hormone), fertilization of the superovulated female (preferably by either breeding with a fertile male or via artificial insemination), introduction of the transgene into the fertilized eggs by known techniques, such as microinjection; implantation of the fertilized eggs into a pseudopregnant female rat, who is then brought to term. Once the fetuses in the pseudopregnant female have been brought to term, a founder animal is identified by standard techniques of hybridization of transgene DNA to genomic DNA from weanling offspring or by a PCR assay that is specific for the presence of the transgene. Founders that express the gene, particularly those that express the gene at levels and with the intended tissue distribution (such as brain specific expression) are selected and bred to establish the intended line or lines of transgenic rats.

It will be appreciated upon practicing the present invention that not all transgenic animals which have an incorporated human B₁ bradykinin gene will exhibit appropriate expression of the B, genes of interest. For instance, data presented in Examples 3 and 6 show variable binding of ³H-DALK to the human B₁ bradykinin receptor on three separate transgenic rat lines expressing the human B₁ bradykinin receptor. Identifying an appropriate transgenic line may also be construct specific, such a differences in promoter strength, number of transgenes incorporated into the genome, as well as the location of these integration events. The rat B₁ receptor is normally expressed at a much lower level than the transgene but its expression can be induced by certain treatments, e.g. lipopolysaccharide or streptozocin. As shown herein when comparing transgenic to non-transgenic rats, the rat B₁ bradykinin receptor has pharmacological properties that are distinct relative to the human receptor, i.e. many synthetic compounds that have high affinity for the human B₁ receptor have low affinity for the rat B₁ receptor. Animals which express the transgene at sufficient amounts under normal conditions are especially useful in receptor occupancy assays. Animals which have expression levels similar to or greater than line 0004, as measured in whole tissue assays, are preferred. However, lines with lower tissue expression (such as lines 0014 and 0015) may be useful if, for example, expression is localized within a discrete region of the tissue which is amenable to further study. To this end, one of ordinary skill in the art can expect to generate from about 6 to about 10 or so lines to be ensured that at least one of the resultant lines will exhibit the desired trait. It may also be useful to identify and breed animals which have multiple copies of the human B₁ bradykinin incorporated into the target genome, such as from 2 to about 50 copies of the selected transgene. Therefore, it is within the purview of the present invention to characterize a specific transgenic animal to find a best fit for in vivo/ex vivo assays to determine binding and/or receptor occupancy characteristics of for a specific test compound, wherein a specific binding/pharmacological profile will exist for the test compound in regard to the native and transgenic B₁ bradykinin receptor protein.

The nomenclature used herein and the laboratory procedures in transgenic protocols, cell culture, molecular genetics, and molecular biology are well known and commonly employed in the art. Standard techniques are used for recombinant nucleic acid methods, polynucleotide synthesis, cell culture, and transgene incorporation (e.g., electroporation, microinjection, lipofection). Various enzymatic reactions, oligonucleotide synthesis, and purification steps are performed according to the manufacturer's specifications. The techniques and procedures are generally performed according to conventional methods in the art and various general references which are provided throughout this document. The procedures therein are believed to be well known in the art and are provided for the convenience of the reader. All the information contained therein is incorporated herein by reference. The following examples are presented by the way of illustration and, because various other embodiments will be apparent to those in the art, the following is not to be construed as a limitation on the scope of the invention.

### EXAMPLE 1

Construction of Transgenic Targeting Vectors

Construct #1—ratEnolase intronA hB1 polyA2 vector

[Step 1]—Rat genomic DNA (50 or 100 ng/50 ul reaction) was used as a template to generate a PCR fragment comprising the rat neuron specific enolase promoter region. Thirty two cycles of PCR were performed (94° C. 25 sec, 60° C. 25 sec, 68° C. 3 min) with Expand High fidelity polymerase (Roche). The forward primer was: Rat_enl.2f: 5′-CATCACTGAGCCCAACACAA-3′ (SEQ ID NO:5) and the reverse primer was Rat_enl.2r: 5′-TCACCTCGAGGACTGCAGAC-3′ (SEQ ID NO:6). This PCR product was 2059 bp in length.

[Step 2]—The purified PCR product (Qiaquick PCR purification) from Step 1 was used as a template for a second round of PCR to add a BamHI restriction site. Thirty cycles of PCR (94° C. 25 sec, 60° C. 25 sec, 68° C. 3 min) with Expand High fidelity polymerase using the following primers: Forward Rat_enl.4f, 5′-GCGGATCCTGAGCTCCTCCTCTGCTCGC-3′ (SEQ ID NO:7); Reverse NSE_{—}1R, 5′-CTCGAGGACTGCAGACTCAG-3′ (SEQ ID NO:8). The resulting product is 1814 bp in length.

[Step 3a]—A plasmid DNA template containing the CMVIntron A sequence was used as a template to generate a PCR fragment for subcloning. Twenty five cycles (94° C. 25 sec, 60° C. 25 sec, 72° C. 1 min) were performed using Pfu polymerase (Stratagene). The forward primer was CMVintA.1F: 5′-GTAAGTACCGCCTATAGAGTC-3′ (SEQ ID NO:9) and the reverse primer is CMVintA.1R: 5° CTGCAGAAAAGACCCATGGAAAGG-3′ (SEQ ID NO:10). This PCR product is 827 bp in length.

[Step 3b]—Twelve cycles of PCR were used (94° C. 25 sec, 60° C. 25 sec, 68° C. 1 min 10 sec with either Pfu (Stratagene) or Expand High fidelity polymerase) to add overlap ends to the CMV intron A product of Step 3a. The forward and reverse primers are as follows: Forward:NSE_CMV.OLF1:

(SEQ ID NO: 11)

5′-GAGTCTGCAGTCCTCGAGGTAAGTACCGCCTATAGAGTC-3′;

Reverse CMV_hB1.OLR1: 5′-TGGCGGCGGTACCAAGCTTCTGCAGAAAAGACCCATGGAAAG-3′ (SEQ ID NO:12). This PCR product is 863 bp in length.

[Step 4]—A PCR fragment comprising the human B₁ bradykinin receptor coding sequence plus bovine growth hormone (BGH) polyA signal with overlap ends was constructed via 25 cycles of PCR (94° C. 25 sec, 60° C. 25 sec, 72° C. 3 min) from plasmid pcDNA3 which contains the human bradykinin B1 receptor sequence fused to the BGH poly A sequence. The primers were as follows:

Forward CMV_hB1.OLF1:

(SEQ ID NO: 13)

5′-CTTTCCATGGGTTTTCTGCAGAAGCTTGGTACCGCCGCCA-3′;

Reverse: BGH.1RNot: 5′-GCGCGGCCGCTCCCCAGCATGCCTGCTATTG-3′ (SEQ ID NO: 14). This PCR is 1518 bp in length.

[Step 5]—The CMV intron A was combined with the with human B1_BGH polyA fragments via 25 cycles of PCR (94° C. 25 sec, 60° C. 25 sec, 68° C 4 min 30 sec) using the templates purified from Step 3 and Step 4. The primers were as follows:

Forward NSE_CMV.OLF1:

(SEQ ID NO: 15)

5′-GAGTCTGCAGTCCTCGAGGTAAGTACCGCCTATAGAGTC-3′;

Reverse: BGH.1RNot: 5′-GCGCGGCCGCTCCCCAGCATGCCTGCTATTG-3′ (SEQ ID NO: 14). The PCR product of Step 5 is 2342 bp.

[Step 6]—The PCR product from step 2 was digested with Bam HI and the PCR product from Step 5 was digested with Not I. A three-way ligation was performed with BamHI/NotI digested pCR®-Blunt II-TOPO® vector (Invitrogen).

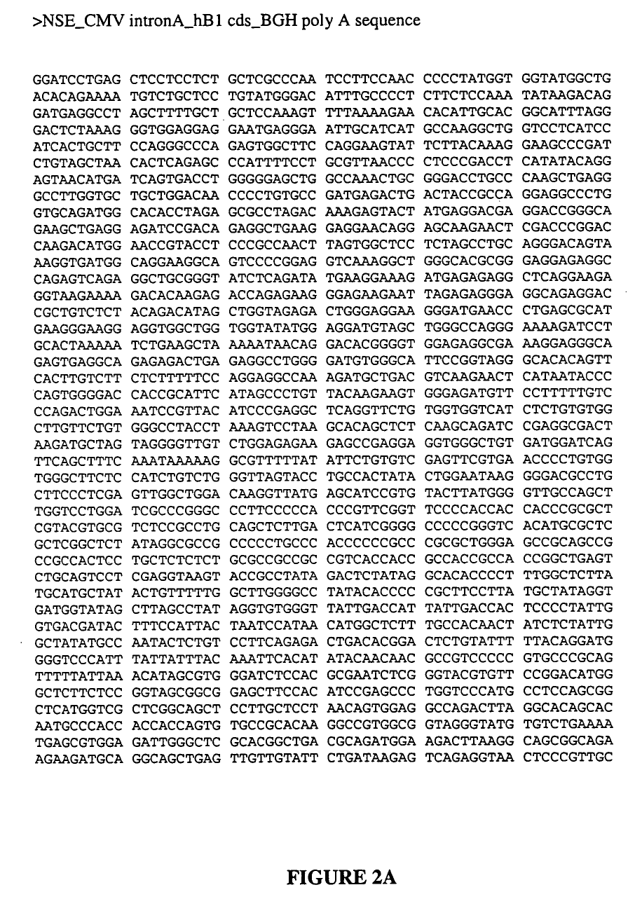

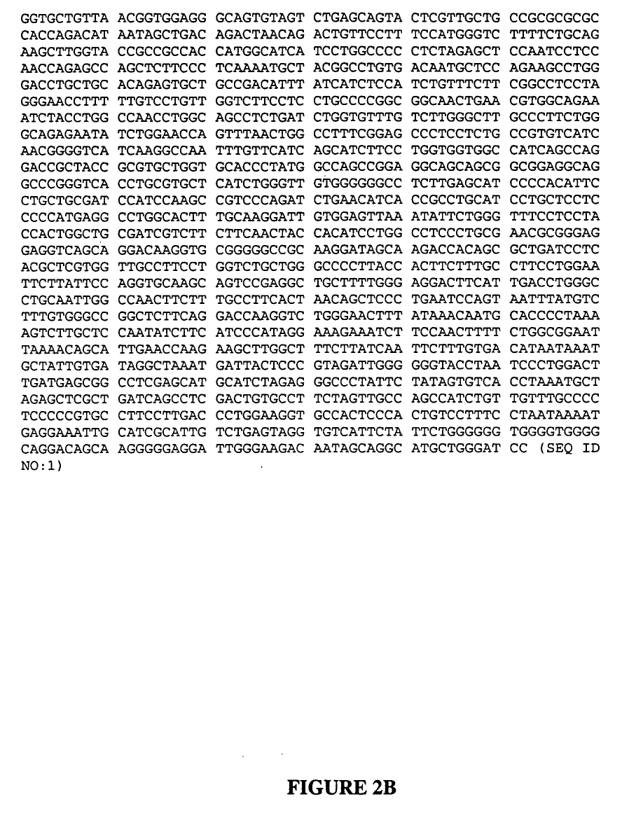

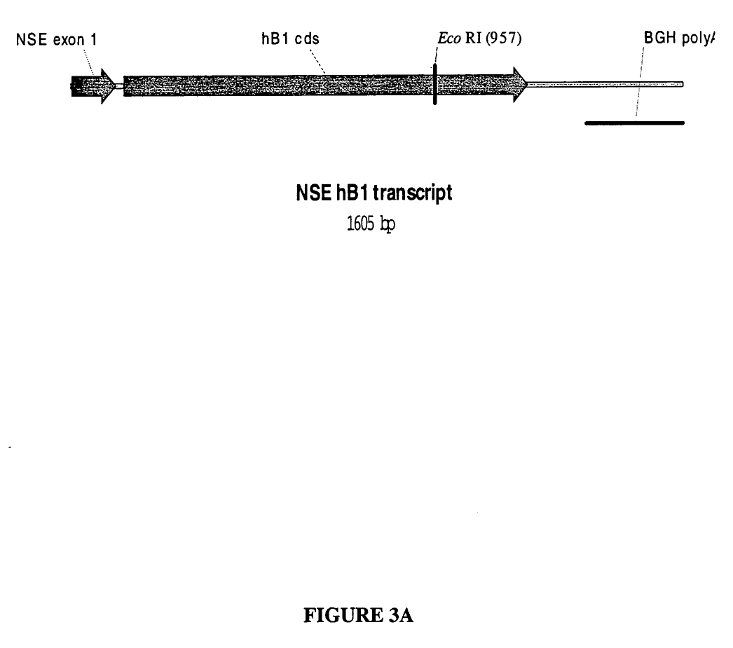

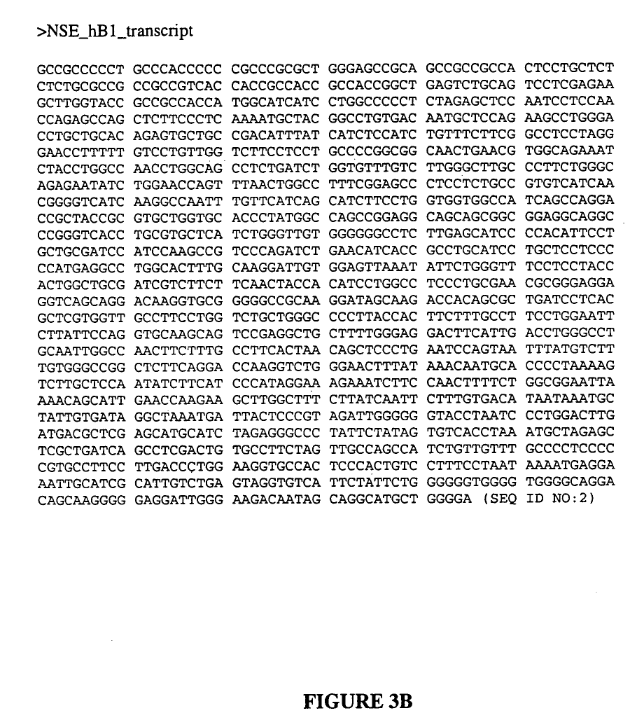

DNA sequence analysis was performed to select clones containing the fewest PCR errors. Selected clones were subcloned into BamHI/NotI digested pBlueScript (pBS) by 3-way ligation with the Bam BI/Afl II 2443 bp fragment and the Afl II/Not I 1699 bp fragment. The resulting transgene is shown in FIG. 1 while the nucleotide sequence of transgene is shown in FIG. 2A-B. A schematic of the transcript for this construct is shown in FIG. 3A while the nucleotide sequence of the projected transcript (shown as a DNA sequence) of the transcript is shown in FIG. 3B.

Construct #2-CMV promoter_CMV intron A_human B1 cds_BGH poly A signal vector

[Step 1—CMV promoter] One hundred nanograms of pcDNA3 was subjected to18 cycles of PCR (94° C. 25 sec, 60° C. 25 sec, 72° C 1 min) with either Pfu or Expand High Fidelity polymerse. The primers were as follows:

Forward CMV promoter 1F:

(SEQ ID NO: 16)

5′-CGGCGGCCGCCGATGTACGGGCCAGATATAC-3′;

Reverse: 5 ′-GACTCTATAGGCGGTACTTACCTATAGTGAGTCGTATTAATTTCG-3′ (SEQ ID NO: 17). The resulting product is 702 bp.

[Step 2—CMV intron A]—One hundred nanograms of a DNA plasmid template comprising the CMV Intron A fragment was subjected to 18 cycles of PCR (94° C. 25 sec, 60° C. 25 sec, 72° C. 1 min) with Pfu polymerase. The primers were as follows:

Forward CMV promoter_intron A 1F 5 ′-CGAAATTAATACGACTCACTATAGGTAAGTACCGCCTATAGAGTC-3′ (SEQ ID NO:18);

Reverse CMVintA.1R 5′-CTGCAGAAAAGACCCATGGAAAGG-3′ (SEQ ID NO: 10). The product is 850 bp in length.

[Step 3] The CMV promoter fragment was linked to the CMV intronA by subjecting the PCR products of Step 1 and Step 2 to 18 cycles of PCR (94° C. 25 sec, 60° C. 25 sec, 72° C. 1 min 30 sec) with Pfu polymerase. The primers were as follows:

Template: 2 ng of each PCR product from step 1 and step 2

Primers: Forward CMV promoter 1F

(SEQ ID NO: 16)

5′-CGGCGGCCGCCGATGTACGGGCCAGATATAC-3′;

Reverse CMVintA.1R 5′-CTGCAGAAAAGACCCATGGAAAGG-3′(SEQ ID NO: 10). This PCR product is 1508 bp in length.

[Step 4]—The CMV promoter_CMV intron A_human B1 bradykinin receptor coding sequence_BGH poly A signal was constructed by digesting the PCR product from Step 3 with Afl II (cuts in CMV intron A). The ratEnolase intronA hB1 polyA2 vector described in this Example was digested with EcoRV and Afl II and these digested fragments were ligated together to generate the transgene shown in FIG. 4.

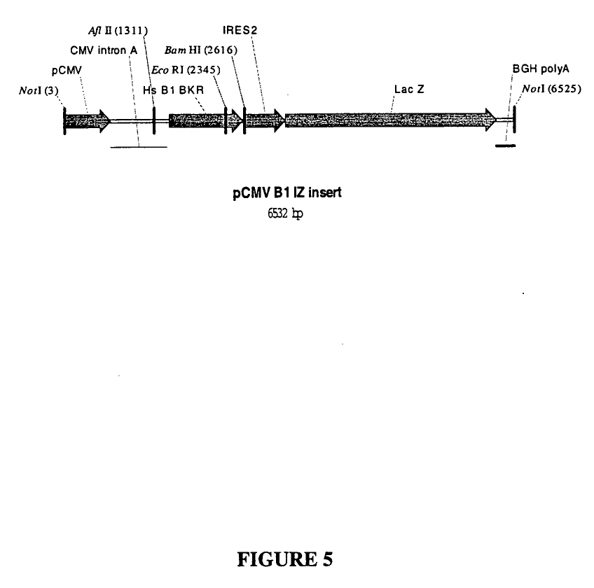

Construct #3—CMV intron A:human B1 coding:IRES element:Lac Z: BGH poly A—The targeting vector as detailed in FIG. 5 was generated as follows:

[Step 1]—The pIRES puro plasmid (Clontech) was used as a template to generate a PCR fragment comprising the IRES element. The PCR reaction was carried out for 20 cycles (94° C. 25 sec, 60° C. 25 sec, 68° C. 1 min 30 sec) with Expand High Fidelity polymerase.

Primers: forward HB1_IRES F

(SEQ ID NO: 19)

5′-CCAACTTTTCTGGCGGAATTAATGCATCTAGGGCGGCCAATTC-3′;

Reverse: IS_LACZ_{—}1R 5 ′-GTAAAACGACGGGATCTATCATGGTGGCGGCGGTTGGCAAGCTTA TCATCGTG-3′ (SEQ ID NO:20). The resulting product is 639 bp in length.

[Step 2]: The LacZ coding region was generated as a PCR fragment by utilizing pcDNA3 beta-Gal plasmid DNA (Invitrogen) as template and running a PCR reaction for 28 cycles (94° C. 25 sec, 60° C. 25 sec, 68° C. 3 min) with Pfu and Expand High Fidelity polymerase. The primers were as follows:

Primers: forward: IS_LACZ_{—}1F

(SEQ ID NO: 21)

5′-CACGATGATAAGCTTGCCAACCGCCGCCACCATGATAGATCCCGTCG
TTTTAC-3′;

Reverse: 5′-GCCTCGAGCTATTTTTGACACCAGACCAACTG-3′ (SEQ ID NO:22). The resulting product is 3098 bp in length.

[Step 3]: The PCR products of Step 1 and Step 2 were linked via 18 cycles of PCR (94° C. 25 sec, 60° C. 25 sec, 68° C. 4 min) with Expand High Fidelity polymerase.

Primers: forward HB1_IRES F

(SEQ ID NO: 19)

5′-CAACTTTTCTGGCGGAATTAATGCATCTAGGGCGGCCAATTC-3′

Reverse: LZ_BGH R 5′-CATTTAGGTGACACTATAGAATCTATTTTTGACACAGACCAACTG-3′ (SEQ ID NO:23). The resulting product is 3721 bp in length.

[Step 4]—The BGH poly A signal is generated by PCR from the plasmid pcDNA3 (Invitrogen) via 18 cycles (94° C. 25 sec, 60° C. 25 sec, 68° C 4 min) with Expand High Fidelity polymerase. The primers are as follows:

Forward LZ_BGH F

(SEQ ID NO: 24)

5′-CAGTTGGTCTGGTGTCAAAAATAGATTCTATAGTGTCACCTAAA
TG-3′;

Reverse: BGH.1RNot

(SEQ ID NO: 14)

5′-GCGCGGCCGCTCCCCAGCATGCCTGCTATTG-3′.

[Step 5]—The BGH polyA PCR fragment of Step 4 was linked to the IRES:LacZ fragment of Step 3 via 20 cycles of PCR (94° C. 25 sec, 60° C. 25 sec, 68° C. 4 min 30 sec) with Expand High Fidelity polymerase. The primers are as follows:

B1_IRES F

(SEQ ID NO: 19)

5′-CCAACTTTTCTGGCGGAATTAATGCATCTAGGGCGGCCAATTC-3′;

Reverse: BGH.1RNot

(SEQ ID NO: 14)

5′-GCGCGGCCGCTCCCCAGCATGCCTGCTATTG-3′

Product: 3963 bp

[Step 6]—The ratEnolase intronA hB1 polyA2 vector described in this Example was used as a template to generate a CMV intron A:human B1 coding sequence via 18 cycles (94° C. 25 sec, 60° C 25 sec, 68° C 2 min 30 sec) of PCR, using the Pfu polymerase. The primers were as follows:

Forward CMVintA.1F—5′-GTAAGTACCGCCTATAGAGTC-3′ (SEQ ID NO:9);

Reverse: HB1_IRES R 5′-GAATTGGCCGCCCTAGATGCATTAATTCCGCCAGAAAAGTTGG-3′ (SEQ ID NO:25). The resulting product is 1931 bp in length.

[Step 7]—The PCR products from Step 6 (CMVintron A: human B1 cds) and Step 1 (IRES) are used as a template to link these to DNA fragments by PCR. Twenty cycles (94° C. 25 sec, 60° C. 25 sec, 68° C. 4 min 30 sec) and Expand High Fidelity polymerase were utilized with the following primers:

Forward: CMVintA.1F

(SEQ ID NO: 9)

5′-GTAAGTACCGCCTATAGAGTC-3′;

Reverse: IS_LACZ_{—}1R 5′-GTAAAACGACGGGATCTATCATGGTGGCGGCGGTTGGCAAGCTTA TCATCGTG-3′ (SEQ ID NO:20). The resulting product is 2549 bp in length.

[Step 8]: The PCR products from Step 7 and Step 5 are used to link the CMVintron A: human B cds (Step 7) to IRES_LacZ_BGH poly A (Step 6) via 18 cycles (94° C. 25 sec, 60° C. 25 sec, 68° C. 7 min 30 sec) of PCR. The primers were as follows:

Forward: CMVintA.1F

(SEQ ID NO: 9)

5′-GTAAGTACCGCCTATAGAGTC-3

Reverse: BGH.1RNot 5′-GCGCGGCCGCTCCCCAGCATGCCTGCTATTG-3′ (SEQ ID NO: 14). The resulting product is 5851 bp in length. This fragment is then subcloned into the DNA expression plasmid pCRII Topo Blunt (Invitrogen) and subjected to DNA sequence analysis to confirm generation of the appropriate transgene.

Construct 4—CMV promoterr:CMV intron A:human B1 coding:IRES2 element:Lac Z: BGH poly A

[Step 1] A 520 bp Bgl II/Nsi I fragment from Construct 3 is subcloned into pIRES2-EGFP (Clontech). This subclone is digested with Bgl II/Nco I. A PCR fragment spanning a portion of LacZ is generated from a pcDNA3_beta Gal (Invitrogen) template via 37 cycles of PCR (94° C. 25 sec, 60° C. 25 sec, 68° C 2 min 30 sec) with Pfu polymerase. The primers were:

Forward: LZ_BspHI

(SEQ ID NO: 26)

5′-GGCATCATGATAGATCCCGTCGTTTTAC-3′;

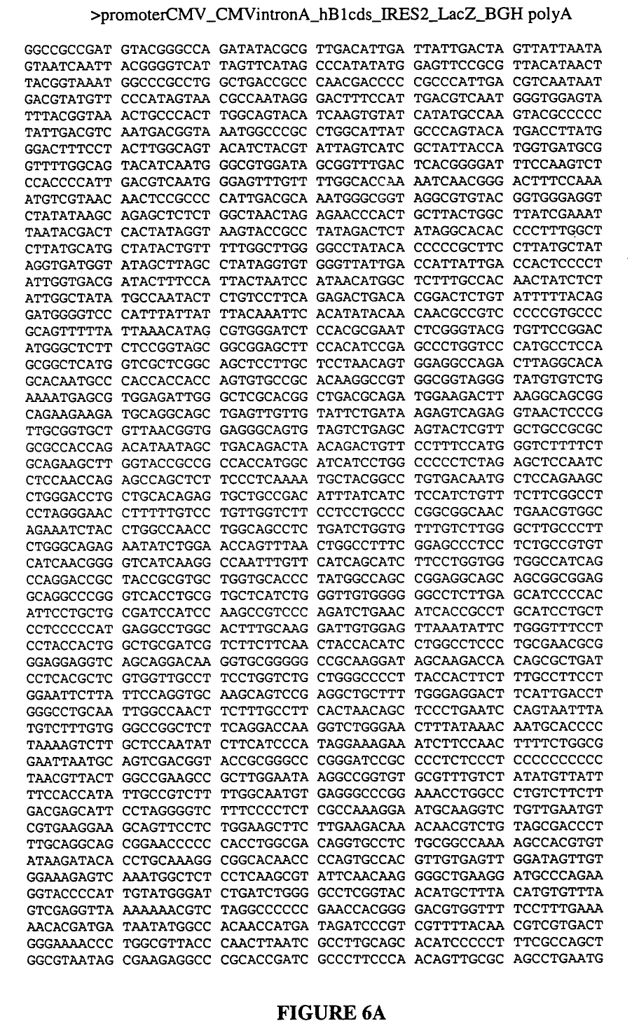

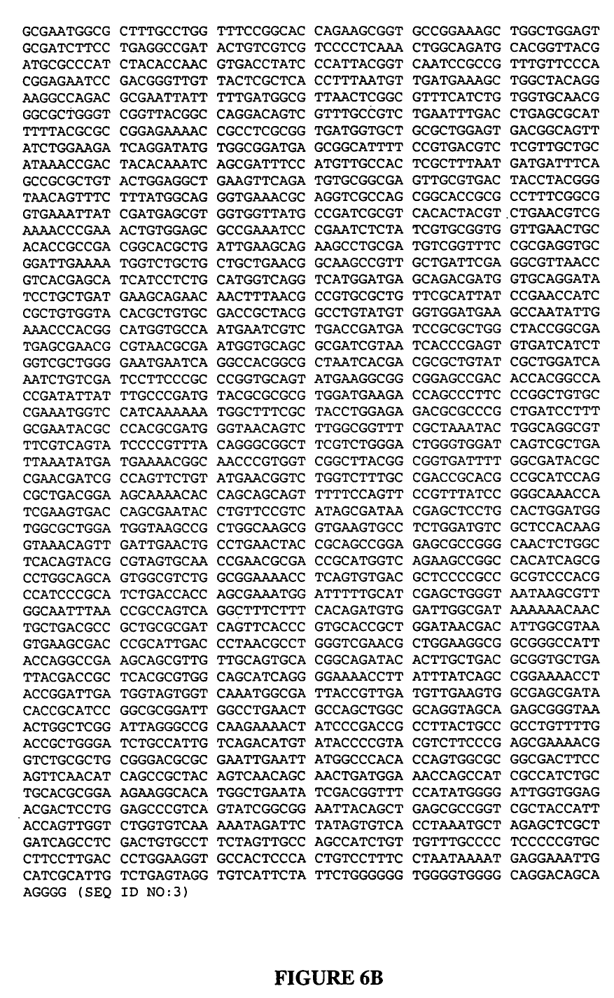

Reverse: 5′-IZ_{—}2699R 5′-TACTGTGAGCCAGAGTTGCC-3′ (SEQ ID NO:27). The resulting product is 2081 bp in length. This product is digested with BspHI and EcoRV. This 1113 bp fragment and the Bgl II/Nco I fragment above are ligated with Construct #2 and digested with Bgl II and EcoRV. The target transgene is shown schematically in FIG. 5 and the nucleotide sequence of the transgene is shown in FIG. 6A-B.

Construct #5—Thy-1 promoter:human B1 coding:IRES2 element:Lac Z: BGH poly A

[Step 1]—A DNA fragment comprising the mouse Thy-1 promoter was generated from a PCR reaction using mouse genomic DNA as a template. The PCR reaction was carried out for 30 cycles (94° C. 25 sec, 60° C. 25 sec, 68° C. 3 min 30 sec) with Expand High Fidelity polymerase. The primers were as follows:

Forward—Thy1_{—}1f_Not:

(SEQ ID NO: 28)

5′-GCGCGGCCGCTCTGGTTATCCAGGCTTCTG-3′;

Reverse—Thy1_hB1r: 5′-GGTGGCGGCGGTACCAAGCTTGTGCCAAGAGTTCCGACTTG-3′ (SEQ ID NO:29). The resulting PCR product is 2923 in length.

[Step 2]—A portion of human B, Bradykinin coding sequence was generated from 10 ng human B₁ receptor cloned into pcDNA3. PCR conditions were as follows: 18 cycles of PCR (94° C. 25 sec, 60° C. 25 sec, 68° C. 4 min) with Pfu polymerase. The primers were:

Forward—Thy1_hB1f:

(SEQ ID NO: 30)

5′-CAAGTCGGAACTCTTGGCACAAGCTTGGTACCGCCGCCACC-3′;

Reverse: hB1_{—}2r 5′-TGCTTGCACCTGGAATAAG-3′ (SEQ ID NO:31). The resulting product was 881 bp in length.

[Step 3]—The PCR products from Step 1 and Step 2 were linked via a PCR reaction (18 cycles @94° C. 25 sec, 60° C. 25 sec, 68° C. 4 min) with Expand High Fidelity polymerase. The primers were:

Forward—Thy1_{—}1f_Not:

(SEQ ID NO: 28)

5′-GCGCGGCCGCTCTGGTTATCCAGGCTTCTG-3′

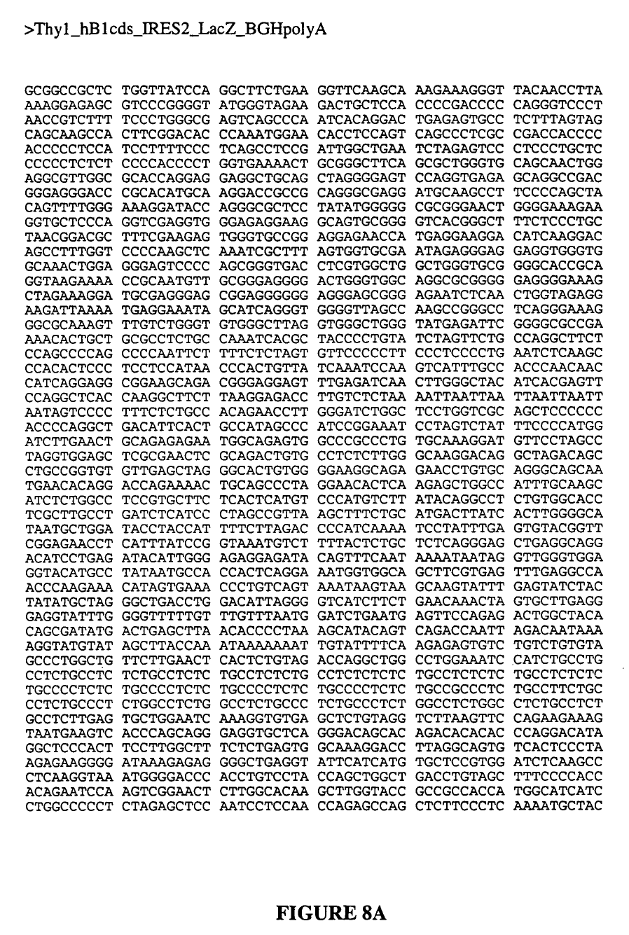

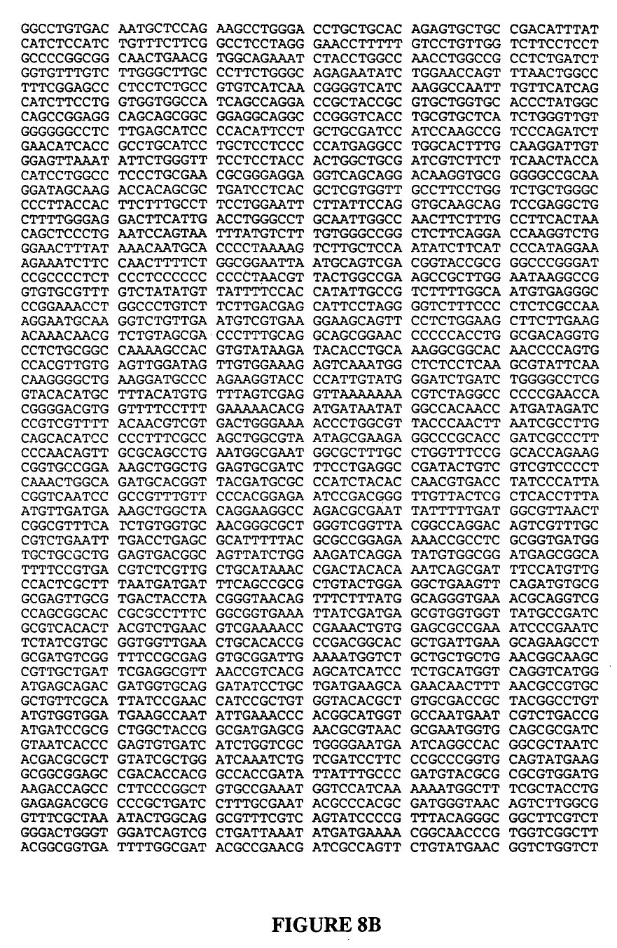

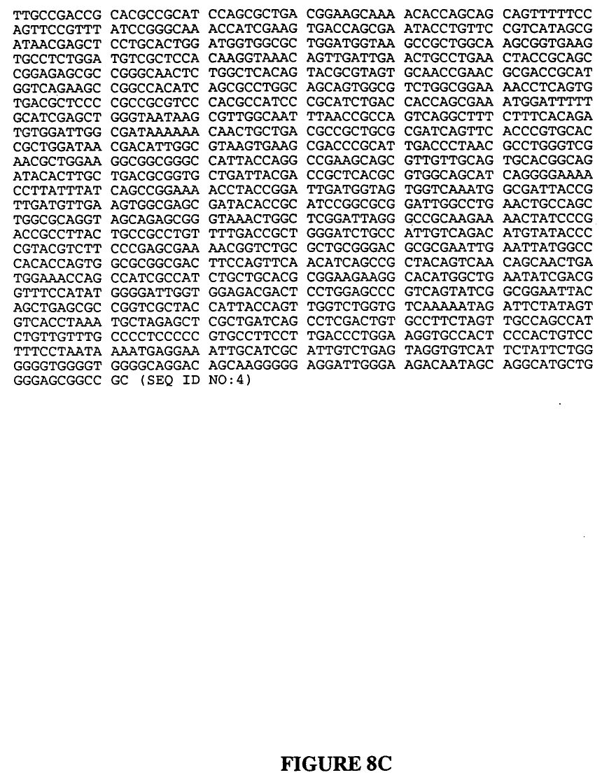

Reverse—hB1_{—}2r: 5′-TGCTTGCACCTGGAATAAG-3′ (SEQ ID NO:32). The resulting product is 3753 bp in length and was cloned into the TOPO TA vector (Invitrogen), followed by DNA sequence analysis of clones.

[Step 4]—The clone from Step 3 is digested with Not I/Bgl II and the 3473 bp fragment is isolated. Construct #4 is digested with Bgl II/Not I and the 4451 bp fragment is isolated. These two fragments are ligated into Not I digested pBlueScript (pBS), resulting the in the transgene disclosed schematically in FIG. 7 and via the nucleotide sequence in FIG. 8A-C.

### EXAMPLE 2

Generation of Transgenic Rats Expressing Human B, Bradykinin 1 Receptor

Approximately 20 ug of NSE promoter CMV intronA_human B1 (FIG. 1) cloned into pBluescript was digested with Bam HI. The 4.1 kb insert was separated from the 3 kb vector on a 0.8% agarose gel. The 4.1 kb band was excised and extracted using Qiaquick Gel Extraction (Qiagen), following extraction the fragment was further purified by separation on a 0.8% agarose gel. The band was excised and extracted from the gel as before with the modification of twice purifying on the Quiquick columns. The final product was resuspended in 10 mM Tris pH 7.4, 0.1 mM EDTA at a concentration of approximately 50 ng/ul. The CMV (FIG. 4) and the Thy-1 promoter constructs (FIG. 7) were prepared in a similar manner with the exception that Not I digestion was used to excise the linear DNA fragment for microinjection from the vector.

A purified NSE promoter_CMV intronA_human B1 (Construct #1, FIG. 1A) fragment was transferred to DNX Transgenic Sciences (Now Xenogen Corporation) in Princeton, N.J. under contract for the generation of transgenic rats containing this transgene. Standard methodology is utilized to transfer said construct into Sprague-Dawley rat eggs to create transgenic rat lines (see, e.g., U.S. Pat. No. 4,873,191) which have incorporated at least one copy of the transgene into the genome. Three such transgenic lines which were transferred and subjected to further genomic and phenotypic analysis were lines 0004, 0014 and 0015. Line 0004 is estimated to have approximately 10 copies, with 0014 having more than line 0004. Of course, while there is a relationship, copy number and expression level are in general poorly correlated.

A Taqman assay was developed for the transcript resulting from transgenic insert containing the NSE promoter_CMV intronA_human B1 bradykinin receptor coding sequence_BGH poly A signal. The splicing of CMV intronA results in a transcript which includes 118 nucleotides of exon 1 of the neuron specific enolase gene fused to the human B1 bradykinin receptor coding sequence (FIG. 3A). PCR primers were designed such that the 3′ end of the forward primers, either NSE_TM1f 5′-GAGTCTGCAGTCCTCGAGAAGC-3′ (SEQ ID NO:33) or NSE_TM2f 5′-TGAGTCTGCAGTCCTCGAGAAG-3′ (SEQ ID NO:34), corresponded to the spliced transcript and therefore would not detect either unspliced transcript or genomic DNA. Taqman probes, NSE_TAQ1, 5′-CTCCAATCCTCCAACCAGAGCCAGC-3′ (SEQ ID NO:35), and NSE_TAQ2,5′-TCCAATCCTCCAACCAGAGCCAGCT-3′ (SEQ ID NO:36)labeled with FAM and TAMRA were designed to detect the PCR products.

An Oligotex Direct mRNA kit (Qiagen) was used to prepare mRNA from the brain of 2 transgenic and 1 non-transgenic rat from line 004. Products derived from the transgenic construct were detected using an ABI PRISM 7700 Sequence Detection System with rodent GAPDH utilized as an internal control. Rodent GAPDH was detected in all samples in contrast the product derived from the transgene was only detected in the transgenic animals. This indicates that the transcript derived from the transgenic insert in line 004 is correctly processed and that this assay can be utilized to distinguish transgenic from non-transgenic animals.

Rat genomic DNA was prepared from tissue by proteinase K digestion followed by phenol chloroform extraction and ethanol precipitation. The genomic DNA (5 to 10 ug) was digested with Eco RI and fragments separated on a 1% agarose gel. DNA was transferred from the gel to Zeta-Probe Genomic Tested blotting membranes (BioRad) using a VacuGene system (Pharmacia Biotech). Pfu polymerase was used to amplify a 701 nucleotide PCR product from the transgenic construct with the forward primer CMV_{—}381F 5′-AATCTCGGGTACGTGTTCCG-3′ (SEQ ID NO:37) and reverse primer Enl_gt2r 5′-TTGGCCAGGTAGATTTCTGC-3′ (SEQ ID NO:38). The product was purified by Qiaquick PCR purification (Qiagen) and radiolabeled with alpha³²PdCTP by random prime labeling (Roche). Hybridization was performed in 0.25 M Na₂HPO₄, 6.5% SDS, and 10% dextran sulfate at 65° C. overnight. The blot washed with a final wash of 0.1×SSC 0.1% SDS for 30 minutes at 60° C. and exposed to film. There is a single Eco RI site in the NSE promoter construct therefore digestion yields a unit length band of 4132 nucleotides, similarly the CMV promoter construct of 6522 contains a single Eco RI site.

### EXAMPLE 3

Ligand Binding to Human B₁ Bradykinin Receptor Purified from Transgenic Rats

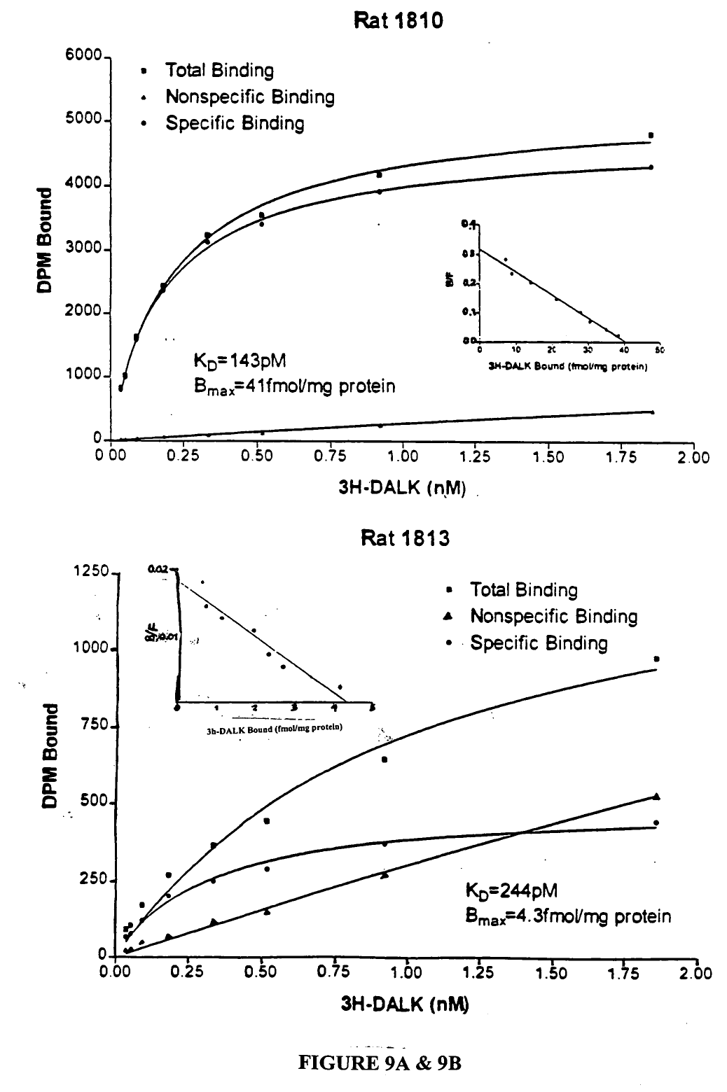

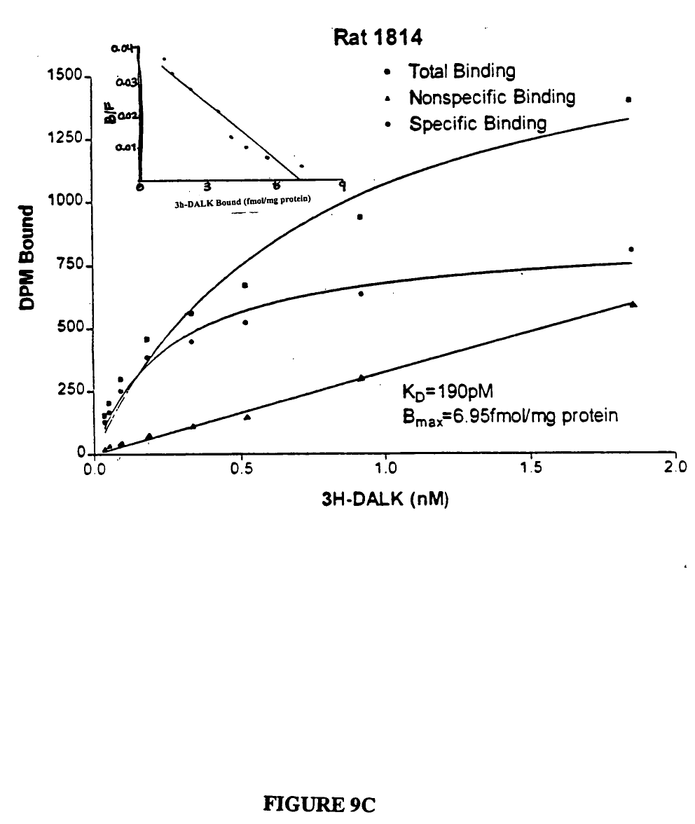

Three of the five lines (0004, 0014, and 0015) of transgenic rats containing Construct #1 (neuron-specific enolase promoter driving expression of the human B₁ bradykinin receptor), as described in Example Section 2, were tested for the ability to bind ³H-DALK, a compound which is approximately 40 fold selective for this ligand. Also important in these assays is the low expression level of endogenous B₁ receptor in neuronal tissue. Briefly, transgenic animals from line 0004, 0014, and 0015 (all females) were decapitated following anesthesia and the whole brain was removed, bisected sagitally and the entire ½ brain weighed. Weights were as follows: line 0004 (813 mg), line 0014 (851 mg), and 0015 (843 mg). The brain tissue was homogenized with a Polytron in ice cold 50 mM Tris-HCl, 1 mM EDTA, 1 mM o-phenanthroline, pH 7.4. The homogenate was centrifuged at 50,000×g for 20 minutes. The pellet was resuspended and homogenized a second time in Tris buffer, and the centrifugation step was repeated. The final pellet was resuspended in assay buffer (20 mM HEPES, 120 mM NaCl, 5 mM KCl, 1 mM o-phenanthroline, 0.2 uM of enaliprilat (the diacid form and active metabolite of enalipril which is added to inhibit angiotensin converting enzyme), 100 μg/ml bacitracin, 3 μM amastatin, 1 μM phosphoramidon, 0.1% BSA, pH 7.4. The assay was carried out in a 0.5 ml volume at room temperature for sixty minutes with 10 mg wet weight tissue/tube. Total protein was determined using a Bio Rad DC assay kit. Specific binding is measured as that which is sensitive to competition with a B, specific ligand, either cold DALK or a compound with specificity for the human B₁ receptor. FIG. 9A, 9B and 9C show measurements of the amount of total, nonspecific and specific binding of ³H-DALK to transgenic rat brain tissue which expresses human B₁ bradykinin receptor. Line 0004 (FIG. 9A) shows expression of 40 fmol/mg protein, Line 0014 (FIG. 9B) shows expression of 4 fmol/mg protein, while Line 0015 (FIG. 9C) shows expression of 7 fmol/mg protein. In contrast, no B₁ receptor is detected in the brains of non-transgenic rats. The Ki values determined for three standard lead compounds in Line 0004 are very similar to those obtained at the cloned hB₁ receptor expressed in CHO cells. Therefore, expression of the human B₁ bradykinin receptor in Line 0004 has the properties expected for the human B₁ receptor.

Line 0004 was subjected to autoradiographic study of the expression of human bradykinin B₁ receptor in transgenic rat brain and spinal cord. A transgenic rat (line 0004) was first anesthetized, and then the brain was removed and immediately frozen on dry ice. The coronal sections (20 μm) of the brain were prepared in a cryostat. The adjacent sections of selected brain regions were divided into two sets and pre-incubated for 15 minutes at room temperature (RT) in buffer A. Following pre-incubation, two sets of the brain sections were incubated separately in buffer B for 90 min at RT. One set of the sections was incubated with 0.3 nM of [³H]DALK, and another set was incubated with both 0.3 nM of [³ H]DALK and 200 μM of unlabeled L-864747. At the end of the incubation period, sections were washed three times (4 min each) in ice-cold buffer A and then rinsed in ice-cold deionized water for five seconds. Sections were dried by cold air at room temperature, then placed in a cassette against Fuji Imaging Plate (BAS-TR2025) at room temperature for a week. The plate was scanned with Fuji BAS-5000 machine, and the images were analyzed using the MCID M5 software (Imaging Research Inc.). Buffer A is 50 mM Tris-HCl, pH 7.5, 120 mM NaCl, 5 mM KCl and Buffer B is 50 mM Tris-HCl, pH 7.5, 120 mM NaCl, 5 mM KCl, 100 μg/ml Bacitracin, Sigma B-0125, 1 μM Phosphoramidon, Sigma R-7385, 1 mM o-Phenanthroline, Sigma P-9375, 3 μM Amastatin, Sigma A-1276, 0.1% BSA (Sigma A-7030). [³ H]DALK is purchased from NEN Life Science (Cat.# NET1096).

The purpose of this autoradiographic study is to characterize human bradykinin B₁ receptor expression in the spinal cord and brain tissues of the transgenic rat carrying human bradykinin B₁ receptor gene by autoradiography. The radiotracer, [³H]DALK for the B₁ receptor was employed in the study and an antagonist of human bradykinin B₁ receptor was used to block the receptor specific binding of [³H]DALK. A signal that was not competed by the antagonist was defined as nonspecific binding of [³H]DALK. The results of autoradiographic study demonstrate expression of human bradykinin B₁ receptor in the brain and spinal cord of the transgenic rat. In NSE_human B₁ receptor transgenic line 0004, the expression of human bradykinin B₁ receptor varies among the different regions of the brain and spinal cord examined. The highest binding signals for [³ H]DALK in transgenic rats are in the dorsal horn of the spinal cord, the cerebral cortex, hypothalamus, thalamus, cerebellum, substantial nigra, interpeduncular nucleus, nucleus of solitary tract, periaqueductal gray, and pontine nucleus. In contrast, [³H]DALK did not show any specific binding signal in the corresponding regions of the brain and spinal cord of the non-transgenic rats, showing that integration of the human B₁ bradykinin gene into the rat genome confers a phenotype of non-native, selective binding characteristics to various test compounds and known modulators of the human B₁ bradykinin receptor.

### EXAMPLE 4

Mapping of the transgene integration site for NSE-hB1 line 0004

Genomic DNA was prepared from tissue of a transgenic rat from line 0004. The genomic DNA was partially digested with restriction endonuclease Sau 3A1 and cloned into the superCOS I vector according to the manufacturer's instructions (Stratagene, La Jolla Calif.). Cosmid clones were screened by standard in situ hybridization of bacterial colonies using a radiolabeled probe consisting of 701 nucleotides. The probe was obtained using standard PCR conditions with the primers, 5′-AATCTCGGGTACGTGTTCCG 3′ (SEQ ID NO:39) and 5′ -TTGGCCAGGTAGATTTCTGC 3′ (SEQ ID NO:40), and the NSE-hB1 transgene construct as the template. Positive colonies were re-screened and cosmid DNA was prepared from clones that were positive in the secondary screen. Cosmid end sequencing was performed using T3 and T7 primers. DNA sequence of cosmid clone 19 that was obtained with the T3 primer was found to match rat genomic DNA containing a portion of the pellucidae glycoprotein gene 1 (ZP-1), whereas the sequence from the T7 primer matched a portion of the NSE_hB1 transgene construct. To fine map the transgene integration site, cosmid 19 was digested with the restriction endonuclease DraI, and the resulting fragments were sub-cloned into the vector pBluescript II (Stratatene, La Jolla, Calif.). Plasmid DNA was prepared from ampicillin resistant colonies and the size of the insert was determined, clones with various size inserts were analyzed by DNA sequence analysis using m13 forward and reverse primers. DNA sequence analysis of clone Dra37 revealed that it contained rat genomic DNA and a portion of the NSE-hB₁ transgene construct. Thus clone Dra37 contained one end of the transgene insertion site. BioInformatic analysis of rat genomic DNA sequence from Dra37 indicated that it matched the DNA sequence of Rattus norvegicus clone CH230-6B11 (GenBank Accession number AC097387). The clone CH230-6B11 contains the zona pellucidae glycoprotein gene 1 (ZP1), the same gene that was identified by end sequencing of cosmid clone 19, and is mapped to chromosome 1. Therefore the transgene integrated into chromosome 1 near the ZP1 gene. The delineation of the transgene insertion site permitted the development of a genotyping assay for identification of the line 0004 homozygous transgenic rats. This randon integration site contains mutliple copies of the transgene. The sequence of clone Dra 37 containing the line 0004 transgene insertion site is as follows:

(SEQ ID NO: 41)
GTACCCTGCC TCCGATATTT GTTAGAACAA CGGTTCCCCG CCACCTACCA ACTGTTTATG
TTTTCTCTAA CAAAAACCAG ACCGGCCGCT GGGCCTGATA CCTGAGTTCA GTCACCAAGA
CCCACGTGGC AGAAGGAGAG AACTGACTTC TGCATATTAT CCTCCAACAC ACACACACAC
ACACACACAC ACACACACAC ACACACACAC ACACACTAAA ATAAATAAAT AGTCTGGGCT
TGGTGGCACA TTGAGAACTT ACCTCAGAAA AAAGGTAAGT AGATAAAGTA AAACTAAAAT
GGAGTGACTC ACACTGGAGT TCCATGTTAC CAAATTAAAA CTAGCTTTCT GACCTTCTGA
GAAACCAGGA CAGAAAGAGG TGAAGGCCAC ATTTTCTAGC CATGCCAACT GCAGCAAACA
TAACTCTGTT CTGGCTGCCA TTGTCCTTAT GAAAAGTAAG CAGCAGGGAT CTGATCTATT
AACCAGCTAG CTCTGTGCTT CCCTCCTCTT CTCCCAACCT CCCAAGGAAA ACATACTCCG
TCCTTTTCCT TTGTTTTATT CCTGCTTCCT GTCTAGGAAA TCACTCCCCT CCAAGGCGTC
AGAACACATT CTGGCTTACA GAATGAAGTT TTACCCAATT CTAGAATCAC AAAATATAGC
CAACGTAAAC CTTGAATGTG ATCTAATTGG TCTAAGAGGC AGAAATGAGA TGAAGAAAAA
AACTGCCGAC ATAGATTTCA GTCTATGGGA TGATGGGCAC ATAAACAATA AGAAGAAAGT
GCCAGACAGG GGTAGGTGCT CTAAATACAA GATAAATTAG AGCAGGTTGA GAAGATGGTA
CTGGGGATTG GAGGGGCGAC TGCTTTAGGC AGGGTATGGC AAAGGTATGC CCCCTGAGAG
AGGATGTTCA TTTTTAGCAC TTGAATTTTA TTTTAGTGTA TGTGTATGCA TGTGCCACAG
CAAATGTATA GAAGTAAAAG GAGACCTTGA GAGAAGTGGT TCACTCCTCC CATGTTGGTC
TTGGGATCGA AGTCAGGTTG TTAGACTTGA CAGGAAGTTT CTCTCCCCAG TGAGCTGTCT
CACCAGCCCA AACGGTGGCA ACATTTTTGC TGAGACCTAA ATAAAGGACA TGCGTCAGTT
CAGAAACCAC AGATATCTGA TCAACCAAGC TCCTGCAGTC TCACCTCATC TTCCTCTCAG
CCACACTGGC CCTTCAGTGG CCCCAGCAGT CCCCGAGGTA GGTGGCTCAA AATGTTTATG
TGGCTACCTT TCATCAACTC CTTCCCCATC TCCAGCCCCG GCCAGACCCT CCAGGGCAAA
CTGAGGCCTC ATCTGAGCTC CTCCTCTGCT CGCCCAATCC TTCCAACCCC CTATGGTGGT
ATTGTCTGTT TACCCTATAG GACATCCTAT AGGGTAAACA GACAATAGAC CATAGGACAA
CAGGCAGGAG CATGCCTGCT ATTGTCCTCC CTTGTCCTCC CTGCCATCCT AAAGCTGGCA
GGTGGCTGGT GCTATATGGA GGATGTAGCT GGGCCAGGGA AAAGATCCTG CACTAAAAAT
CTGAAGCTAA AAATAACAGG ACACGGGATG GAGGAGCTCA GGTGGTATGG CTGACACAGA
AAATGTCTGC TCCTGTATGG GACATTTGCC CCTCTTCTCC AAATATAAGA CAGGATGAGG
CCTAGCTTTT GCTGCTCCAA AGTTTTA.

### EXAMPLE 5

Development of a Genotype Assay for NSE_HB, line 0004 Homozygous Transgenic Rats

The genomic DNA sequence upstream of the transgene insertion site was utilized to design forward, 5′-GAGGTGAAGGCCACATTTTCTAGC-3′ (SEQ ID NO:42), and reverse 5′-ATGGGGAAGGAGTTGATGAAAGGTAGCC-3′ (SEQ ID NO:43), PCR primers. Using the cosmid DNA template and standard PCR procedures these primers generate a product of 922 nucleotides. This fragment of 922 nucleotides serves an external probe that can be radiolabeled and used in Southern blot analysis to discern wild type from transgenic chromosomes. Accordingly, Southern blot analysis of wild type rat genomic DNA with the restriction endonuclease DraI results in the detection of a single fragment of approximately 3.1 kb with the external probe. In contrast, the digestion of genomic DNA prepared from a rat heterozygous for the transgene results in the detection of two fragments, one of 3.1 kb and a second of approximately 1.6 kb. The 1.6 kb fragment corresponds to the chromosome with the transgene insertion site, whereas the 3.1 kb fragment corresponds to the wild type chromosome. Thus, DraI digestion and Southern blot analysis with the external probe can be used to identify homozygous wild type animals, heterozygous and homozygous transgenics. This was used to identify and establish a homozygous transgenic breeding colony. Significantly, the line 0004 homozygous animals express 2-fold more human B₁ bradykinin receptor in the brain and spinal cord than the heterozygous animals.

### EXAMPLE 6

Ex vivo Receptor Occupancy Assay in NSE hB_transgenic rat

Transgenic rats of either sex are placed in an induction chamber and anesthetized with isoflurane under a Flow Sciences hood. Once anesthetized, the rat is placed on a circulating water warming blanket (Gaymar T-pump) and anesthesia is maintained using 2% isoflurane by means of a nose cone. The tail vein is cannulated with a 25G winged infusion set-up connected to a syringe containing either test compound or vehicle. The desired dose of test compound is administered. At the experimental end-point a blood sample is taken, the rat is euthanized, and tissue is removed (typically brain and spinal cord) for subsequent assays.

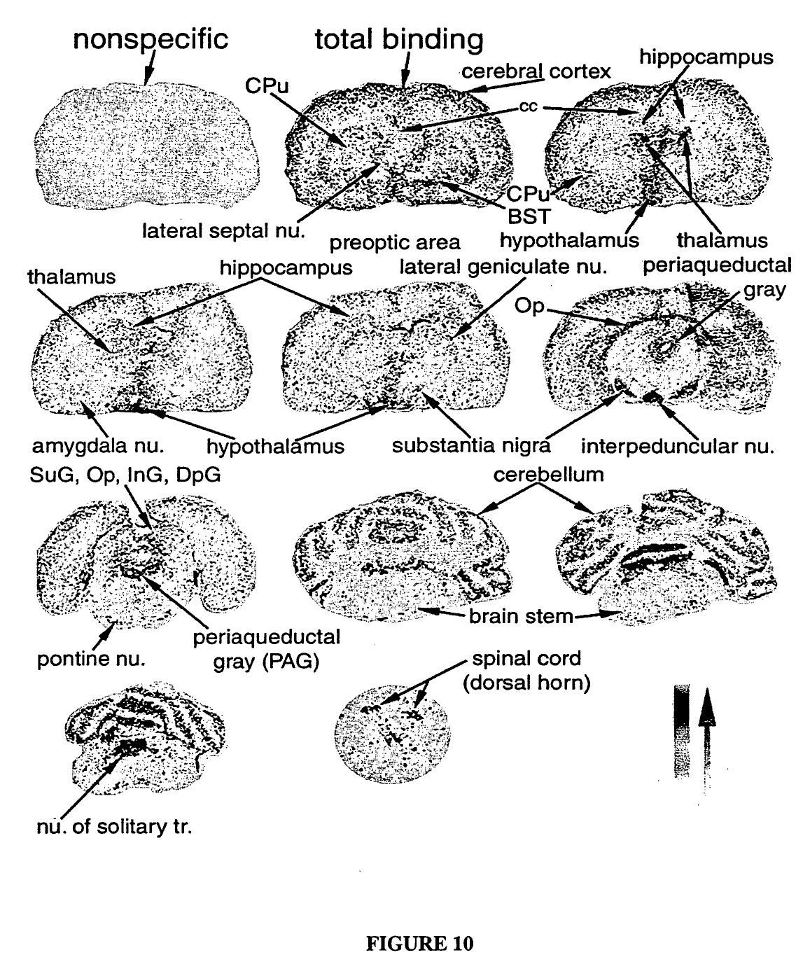

For autoradiographic analysis of human B₁ receptor expression, tissues removed from transgenic rats were frozen on dry ice powder, and stored at −70° C. Coronal sections of the brain and the transverse sections of the spinal cord were prepared with cryostat (Leica, CM3050) at 20 μM of each. The frozen sections were stored at −70° C. For analysis, frozen sections were warmed at room temperature (RT) for 15 minutes, then followed by 15 minutes preincubation in the buffer without radioligand at RT. After preincubation, the sections were transferred to the incubation buffer, and incubated for 90 minutes at RT. Total binding, both non-specific and specific, was determined by incubating in buffer containing 0.3 nM [H-3] DALK. An adjacent section was utilized to determine non-specific binding, which was incubated in buffer containing 0.3 nM [H-3] DALK and 200 nM of a non-peptide receptor antagonist that exhibits high affinity and specificity for the human B₁ bradykinin receptor. Following the 90 minute incubation, the sections were washed three times, 3 minutes each, in buffer, rinsed in DIH₂O for 30 seconds at 4° C., and then dried by air blower at RT. The sections were placed against Fuji imaging plates, and exposed for a week at RT. The plates were scanned with Fuji PhosphorImager BAS 5000, and the images were analyzed with MCID M5 software. FIG. 10 shows autoradiograms of brain and spinal cord sections from NSE_hB₁ line 0004 transgenic rats. Regions of the brain and spinal cord that exhibit high levels of binding are indicated. Specific [H-3] DALK binding (total binding—nonspecific binding) is indicative of the level of human B₁ bradykinin receptor expression. There is no detectable specific binding of [H-3] DALK in non-transgenic control rats.

For homogenate-based binding assay, thirty-five milligrams of frozen brain (cerebral cortex or cerebellum) or spinal cord is homogenized with a Polytron, in a large volume of ice-cold assay buffer (20 mM HEPES, 120 mM NaCl, 5 mM KCl, pH 7.4) and transferred to two chilled centrifuge tubes. To pellet membranes the tubes are centrifuged for 10 minutes at 75,000×g in a rotor pre-cooled to 4° C. The supernatant is discarded and each tube is rinsed with 20 ml ice-cold buffer and then homogenized pellets above in ice-cold assay buffer. The homogenate is pooled and added to a tube containing the radiotracer, 20 pM of a non-peptide human B₁ receptor antagonist that is labeled with ³⁵S, in each tube containing 0.5 ml room temperature assay buffer. Nonspecific binding is determined by adding homogenate to tubes containing the radiotracer and 100 nM of the unlabeled non-peptide human B. receptor antagonist. At set time points (1,2,4,6,8,10 minutes) the contents of three tubes are filtered over individual 25 mm GF/B filters presoaked in 0.05% Triton X-100. The filtration step is performed by adding 4 ml ice-cold assay buffer to each of the three replicate tubes, pouring the contents over the filters, and washing each filter two times with 4 ml ice-cold buffer. A Hoeffer FH 225V filtration manifold is used for the filtration. The nonspecific binding tubes are similarly filtered after finishing the 6 time points. Filters are transferred to 5ml scintillation vials and counted after soaking 10 hours in 3ml Beckman Ready Safe scintillation fluid.

The specific binding is calculated at each time point (total cpm—nonspecific cpm) and the slope of the association is determined by linear regression. Receptor occupancy in a drug treated animal is determined by the following equation:
% Occupancy=(1-(slope_{drug}/slope_{vehicle}))×100
slope_{drug} is the slope of the association rate line from a drug treated animal. slope_{vehicle} is the slope determined for a vehicle treated animal.

Various patent and journal publications are cited herein, the disclosures of which are all incorporated by reference in their entireties.

1-28. (canceled)

29. A transgenic rat having incorporated into its genome at least one copy of a transgene encoding a human B₁ bradykinin receptor gene, such that the transgenic rodent demonstrates a phenotype associated with a human B₁ bradykinin receptor occupancy or binding profile.

30. The transgenic rat of claim 29 wherein said transgene is operatively linked to a heterologous promoter which effects expression of a human B₁ bradykinin receptor gene.

31. The transgenic rat of claim 30 wherein said heterologous promoter effects neuron specific expression of human B₁ bradykinin receptor protein within the central nervous system of the transgenic rodent.

32. The transgenic rat of claim 31 wherein said heterologous promoter is the rat neuronal specific enolase (NSE) promoter.

33. The transgenic rat of claim 30 wherein said heterologous promoter effects brain specific expression of human B₁ bradykinin receptor protein.

34. The transgenic rat of claim 33 wherein said heterologous promoter is the mouse Thy-1 promoter.

35. The transgenic rat of claim 30 wherein said transgene is a discistronic transgene which further comprises a reporter gene expressed at detectable levels within the transgenic rat.

36. The transgenic rat of claim 35 wherein said transgene is expressed from a heterologous promoter.

37. The transgenic rat of claim 36 wherein said heterologous promoter is selected from the group consisting of the rat neuron specific enolase (NSE) promoter, the cytomegaolvirus (CMV) promoter and the mouse Thy-1 promoter.

38. A transgenic rat of claim 37 wherein the transgene is NSE_CMV intronA_hB1 cds_BGH poly A.

39. A transgenic rat of claim 37 wherein the transgene is CMV_CMV intronA_hB I cds_IRES2_LacZ_BGH polyA.

40. A transgenic rat of claim 37 wherein the transgene is Thy1_hB1cds_IRES2_LacZ_BGHpolyA.

41. A cell isolated from the transgenic rat of claim 29 wherein said cell comprises a transgene encoding a human B₁ bradykinin receptor protein.

42. A cell isolated from the transgenic rat of claim 30 wherein said cell comprises a transgene encoding a human B₁ bradykinin receptor protein.


SEQUENCE LISTING (41 sequences)

SEQ ID NO: 1 - DNA, 4132 bp, human
ggatcctgagctcctcctctgctcgcccaatccttccaaccccctatggtggtatggctgacacagaaaatgtctgctcctgtatgggacatttgcccctcttctccaaatataagacaggatgaggcctagcttttgctgctccaaagttttaaaagaacacattgcacggcatttagggactctaaagggtggaggaggaatgagggaattgcatcatgccaaggctggtcctcatccatcactgcttccagggcccagagtggcttccaggaagtattcttacaaaggaagcccgatctgtagctaacactcagagcccattttcctgcgttaacccctcccgacctcatatacaggagtaacatgatcagtgacctgggggagctggccaaactgcgggacctgcccaagctgagggccttggtgctgctggacaacccctgtgccgatgagactgactaccgccaggaggccctggtgcagatggcacacctagagcgcctagacaaagagtactatgaggacgaggaccgggcagaagctgaggagatccgacagaggctgaaggaggaacaggagcaagaactcgacccggaccaagacatggaaccgtacctcccgccaacttagtggctcctctagcctgcagggacagtaaaggtgatggcaggaaggcagtccccggaggtcaaaggctgggcacgcgggaggagaggccagagtcagaggctgcgggtatctcagatatgaaggaaagatgagagaggctcaggaagaggtaagaaaagacacaagagaccagagaagggagaagaattagagagggaggcagaggaccgctgtctctacagacatagctggtagagactgggaggaagggatgaaccctgagcgcatgaagggaaggaggtggctggtggtatatggaggatgtagctgggccagggaaaagatcctgcactaaaaatctgaagctaaaaataacaggacacggggtggagaggcgaaaggagggcagagtgaggcagagagactgagaggcctggggatgtgggcattccggtagggcacacagttcacttgtcttctctttttccaggaggccaaagatgctgacgtcaagaactcataataccccagtggggaccaccgcattcatagccctgttacaagaagtgggagatgttcctttttgtcccagactggaaatccgttacatcccgaggctcaggttctgtggtggtcatctctgtgtggcttgttctgtgggcctacctaaagtcctaagcacagctctcaagcagatccgaggcgactaagatgctagtaggggttgtctggagagaagagccgaggaggtgggctgtgatggatcagttcagctttcaaataaaaaggcgtttttatattctgtgtcgagttcgtgaacccctgtggtgggcttctccatctgtctgggttagtacctgccactatactggaataaggggacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgaggtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagaagcttggtaccgccgccaccatggcatcatcctggccccctctagagctccaatcctccaaccagagccagctcttccctcaaaatgctacggcctgtgacaatgctccagaagcctgggacctgctgcacagagtgctgccgacatttatcatctccatctgtttcttcggcctcctagggaacctttttgtcctgttggtcttcctcctgccccggcggcaactgaacgtggcagaaatctacctggccaacctggcagcctctgatctggtgtttgtcttgggcttgcccttctgggcagagaatatctggaaccagtttaactggcctttcggagccctcctctgccgtgtcatcaacggggtcatcaaggccaatttgttcatcagcatcttcctggtggtggccatcagccaggaccgctaccgcgtgctggtgcaccctatggccagccggaggcagcagcggcggaggcaggcccgggtcacctgcgtgctcatctgggttgtggggggcctcttgagcatccccacattcctgctgcgatccatccaagccgtcccagatctgaacatcaccgcctgcatcctgctcctcccccatgaggcctggcactttgcaaggattgtggagttaaatattctgggtttcctcctaccactggctgcgatcgtcttcttcaactaccacatcctggcctccctgcgaacgcgggaggaggtcagcaggacaaggtgcgggggccgcaaggatagcaagaccacagcgctgatcctcacgctcgtggttgccttcctggtctgctgggccccttaccacttctttgccttcctggaattcttattccaggtgcaagcagtccgaggctgcttttgggaggacttcattgacctgggcctgcaattggccaacttctttgccttcactaacagctccctgaatccagtaatttatgtctttgtgggccggctcttcaggaccaaggtctgggaactttataaacaatgcacccctaaaagtcttgctccaatatcttcatcccataggaaagaaatcttccaacttttctggcggaattaaaacagcattgaaccaagaagcttggctttcttatcaattctttgtgacataataaatgctattgtgataggctaaatgattactcccgtagattggggggtacctaatccctggacttgatgagcggcctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggatcc

SEQ ID NO: 2 - DNA, 1605 bp, human
gccgccccctgcccaccccccgcccgcgctgggagccgcagccgccgccactcctgctctctctgcgccgccgccgtcaccaccgccaccgccaccggctgagtctgcagtcctcgagaagcttggtaccgccgccaccatggcatcatcctggccccctctagagctccaatcctccaaccagagccagctcttccctcaaaatgctacggcctgtgacaatgctccagaagcctgggacctgctgcacagagtgctgccgacatttatcatctccatctgtttcttcggcctcctagggaacctttttgtcctgttggtcttcctcctgccccggcggcaactgaacgtggcagaaatctacctggccaacctggcagcctctgatctggtgtttgtcttgggcttgcccttctgggcagagaatatctggaaccagtttaactggcctttcggagccctcctctgccgtgtcatcaacggggtcatcaaggccaatttgttcatcagcatcttcctggtggtggccatcagccaggaccgctaccgcgtgctggtgcaccctatggccagccggaggcagcagcggcggaggcaggcccgggtcacctgcgtgctcatctgggttgtggggggcctcttgagcatccccacattcctgctgcgatccatccaagccgtcccagatctgaacatcaccgcctgcatcctgctcctcccccatgaggcctggcactttgcaaggattgtggagttaaatattctgggtttcctcctaccactggctgcgatcgtcttcttcaactaccacatcctggcctccctgcgaacgcgggaggaggtcagcaggacaaggtgcgggggccgcaaggatagcaagaccacagcgctgatcctcacgctcgtggttgccttcctggtctgctgggccccttaccacttctttgccttcctggaattcttattccaggtgcaagcagtccgaggctgcttttgggaggacttcattgacctgggcctgcaattggccaacttctttgccttcactaacagctccctgaatccagtaatttatgtctttgtgggccggctcttcaggaccaaggtctgggaactttataaacaatgcacccctaaaagtcttgctccaatatcttcatcccataggaaagaaatcttccaacttttctggcggaattaaaacagcattgaaccaagaagcttggctttcttatcaattctttgtgacataataaatgctattgtgataggctaaatgattactcccgtagattggggggtacctaatccctggacttgatgacgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 3 - DNA, 6485 bp, human
ggccgccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataggtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagaagcttggtaccgccgccaccatggcatcatcctggccccctctagagctccaatcctccaaccagagccagctcttccctcaaaatgctacggcctgtgacaatgctccagaagcctgggacctgctgcacagagtgctgccgacatttatcatctccatctgtttcttcggcctcctagggaacctttttgtcctgttggtcttcctcctgccccggcggcaactgaacgtggcagaaatctacctggccaacctggcagcctctgatctggtgtttgtcttgggcttgcccttctgggcagagaatatctggaaccagtttaactggcctttcggagccctcctctgccgtgtcatcaacggggtcatcaaggccaatttgttcatcagcatcttcctggtggtggccatcagccaggaccgctaccgcgtgctggtgcaccctatggccagccggaggcagcagcggcggaggcaggcccgggtcacctgcgtgctcatctgggttgtggggggcctcttgagcatccccacattcctgctgcgatccatccaagccgtcccagatctgaacatcaccgcctgcatcctgctcctcccccatgaggcctggcactttgcaaggattgtggagttaaatattctgggtttcctcctaccactggctgcgatcgtcttcttcaactaccacatcctggcctccctgcgaacgcgggaggaggtcagcaggacaaggtgcgggggccgcaaggatagcaagaccacagcgctgatcctcacgctcgtggttgccttcctggtctgctgggccccttaccacttctttgccttcctggaattcttattccaggtgcaagcagtccgaggctgcttttgggaggacttcattgacctgggcctgcaattggccaacttctttgccttcactaacagctccctgaatccagtaatttatgtctttgtgggccggctcttcaggaccaaggtctgggaactttataaacaatgcacccctaaaagtcttgctccaatatcttcatcccataggaaagaaatcttccaacttttctggcggaattaatgcagtcgacggtaccgcgggcccgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatgatagatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacctttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccggccacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgacgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggagacgactcctggagcccgtcagtatcggcggaattacagctgagcgccggtcgctaccattaccagttggtctggtgtcaaaaatagattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggg

SEQ ID NO: 4 - DNA, 7932 bp, human
gcggccgctctggttatccaggcttctgaaggttcaagcaaagaaagggttacaaccttaaaaggagagcgtcccggggtatgggtagaagactgctccaccccgacccccagggtccctaaccgtcttttccctgggcgagtcagcccaatcacaggactgagagtgcctctttagtagcagcaagccacttcggacacccaaatggaacacctccagtcagccctcgccgaccaccccaccccctccatccttttccctcagcctccgattggctgaatctagagtccctccctgctcccccctctctccccacccctggtgaaaactgcgggcttcagcgctgggtgcagcaactggaggcgttggcgcaccaggaggaggctgcagctaggggagtccaggtgagagcaggccgacgggagggacccgcacatgcaaggaccgccgcagggcgaggatgcaagccttccccagctacagttttgggaaaggataccagggcgctcctatatgggggcgcgggaactggggaaagaaggtgctcccaggtcgaggtgggagaggaaggcagtgcggggtcacgggctttctccctgctaacggacgctttcgaagagtgggtgccggaggagaaccatgaggaaggacatcaaggacagcctttggtccccaagctcaaatcgctttagtggtgcgaatagagggaggaggtgggtggcaaactggagggagtccccagcgggtgacctcgtggctggctgggtgcggggcaccgcaggtaagaaaaccgcaatgttgcgggaggggactgggtggcaggcgcgggggaggggaaagctagaaaggatgcgagggagcggaggggggagggagcgggagaatctcaactggtagaggaagattaaaatgaggaaatagcatcagggtggggttagccaagccgggcctcagggaaagggcgcaaagtttgtctgggtgtgggcttaggtgggctgggtatgagattcggggcgccgaaaacactgctgcgcctctgccaaatcacgctacccctgtatctagttctgccaggcttctccagccccagccccaattcttttctctagtgttcccccttccctcccctgaatctcaagcccacactccctcctccataacccactgttatcaaatccaagtcatttgccacccaacaaccatcaggaggcggaagcagacgggaggagtttgagatcaacttgggctacatcacgagttccaggctcaccaaggcttcttaaggagaccttgtctctaaaattaattaattaattaattaatagtcccctttctctgccacagaaccttgggatctggctcctggtcgcagctccccccaccccaggctgacattcactgccatagcccatccggaaatcctagtctatttccccatggatcttgaactgcagagagaatggcagagtggcccgccctgtgcaaaggatgttcctagcctaggtggagctcgcgaactcgcagactgtgcctctcttgggcaaggacaggctagacagcctgccggtgtgttgagctagggcactgtggggaaggcagagaacctgtgcagggcagcaatgaacacaggaccagaaaactgcagccctaggaacactcaagagctggccatttgcaagcatctctggcctccgtgcttctcactcatgtcccatgtcttatacaggcctctgtggcacctcgcttgcctgatctcatccctagccgttaagctttctgcatgacttatcacttggggcataatgctggatacctaccattttcttagaccccatcaaaatcctatttgagtgtacggttcggagaacctcatttatccggtaaatgtcttttactctgctctcagggagctgaggcaggacatcctgagatacattgggagaggagatacagtttcaataaaataataggttgggtggaggtacatgcctataatgccaccactcaggaaatggtggcagcttcgtgagtttgaggccaacccaagaaacatagtgaaaccctgtcagtaaataagtaagcaagtatttgagtatctactatatgctagggctgacctggacattaggggtcatcttctgaacaaactagtgcttgagggaggtatttggggtttttgtttgtttaatggatctgaatgagttccagagactggctacacagcgatatgactgagcttaacacccctaaagcatacagtcagaccaattagacaataaaaggtatgtatagcttaccaaataaaaaaattgtattttcaagagagtgtctgtctgtgtagccctggctgttcttgaactcactctgtagaccaggctggcctggaaatccatctgcctgcctctgcctctctgcctctctgcctctctgcctctctctctgcctctctctgcctctctctgcccctctctgcccctctctgcccctctctgcccctctctgccgccctctgccttctgccctctgccctctggcctctggcctctgccctctgccctctggcctctggcctctgcctctgcctcttgagtgctggaatcaaaggtgtgagctctgtaggtcttaagttccagaagaaagtaatgaagtcacccagcagggaggtgctcagggacagcacagacacacacccaggacataggctcccacttccttggctttctctgagtggcaaaggaccttaggcagtgtcactccctaagagaaggggataaagagaggggctgaggtattcatcatgtgctccgtggatctcaagccctcaaggtaaatggggacccacctgtcctaccagctggctgacctgtagctttccccaccacagaatccaagtcggaactcttggcacaagcttggtaccgccgccaccatggcatcatcctggccccctctagagctccaatcctccaaccagagccagctcttccctcaaaatgctacggcctgtgacaatgctccagaagcctgggacctgctgcacagagtgctgccgacatttatcatctccatctgtttcttcggcctcctagggaacctttttgtcctgttggtcttcctcctgccccggcggcaactgaacgtggcagaaatctacctggccaacctggccgcctctgatctggtgtttgtcttgggcttgcccttctgggcagagaatatctggaaccagtttaactggcctttcggagccctcctctgccgtgtcatcaacggggtcatcaaggccaatttgttcatcagcatcttcctggtggtggccatcagccaggaccgctaccgcgtgctggtgcaccctatggccagccggaggcagcagcggcggaggcaggcccgggtcacctgcgtgctcatctgggttgtggggggcctcttgagcatccccacattcctgctgcgatccatccaagccgtcccagatctgaacatcaccgcctgcatcctgctcctcccccatgaggcctggcactttgcaaggattgtggagttaaatattctgggtttcctcctaccactggctgcgatcgtcttcttcaactaccacatcctggcctccctgcgaacgcgggaggaggtcagcaggacaaggtgcgggggccgcaaggatagcaagaccacagcgctgatcctcacgctcgtggttgccttcctggtctgctgggccccttaccacttctttgccttcctggaattcttattccaggtgcaagcagtccgaggctgcttttgggaggacttcattgacctgggcctgcaattggccaacttctttgccttcactaacagctccctgaatccagtaatttatgtctttgtgggccggctcttcaggaccaaggtctgggaactttataaacaatgcacccctaaaagtcttgctccaatatcttcatcccataggaaagaaatcttccaacttttctggcggaattaatgcagtcgacggtaccgcgggcccgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatgatagatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacctttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccggccacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgacgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggagacgactcctggagcccgtcagtatcggcggaattacagctgagcgccggtcgctaccattaccagttggtctggtgtcaaaaatagattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagcggccgc

SEQ ID NO: 5 - DNA, 20 bp, Artificial Sequence
  oligonucleotide
catcactgagcccaacacaa

SEQ ID NO: 6 - DNA, 20 bp, Artificial Sequence
  oligonucleotide
tcacctcgaggactgcagac

SEQ ID NO: 7 - DNA, 28 bp, Artificial Sequence
  oligonucleotide
gcggatcctgagctcctcctctgctcgc

SEQ ID NO: 8 - DNA, 20 bp, Artificial Sequence
  oligonucleotide
ctcgaggactgcagactcag

SEQ ID NO: 9 - DNA, 21 bp, Artificial Sequence
  oligonucleotide
gtaagtaccgcctatagagtc

SEQ ID NO: 10 - DNA, 24 bp, Artificial Sequence
  oligonucleotide
ctgcagaaaagacccatggaaagg

SEQ ID NO: 11 - DNA, 39 bp, Artificial Sequence
  oligonucleotide
gagtctgcagtcctcgaggtaagtaccgcctatagagtc

SEQ ID NO: 12 - DNA, 42 bp, Artificial Sequence
  oligonucleotide
tggcggcggtaccaagcttctgcagaaaagacccatggaaag

SEQ ID NO: 13 - DNA, 42 bp, Artificial Sequence
  oligonucleotide
ctttccatgggtcttttctgcagaagcttggtaccgccgcca

SEQ ID NO: 14 - DNA, 31 bp, Artificial Sequence
  oligonucleotide
gcgcggccgctccccagcatgcctgctattg

SEQ ID NO: 15 - DNA, 39 bp, Artificial Sequence; identical to SEQ ID NO: 11
  oligonucleotide

SEQ ID NO: 16 - DNA, 31 bp, Artificial Sequence
  oligonucleotide
cggcggccgccgatgtacgggccagatatac

SEQ ID NO: 17 - DNA, 45 bp, Artificial Sequence
  oligonucleotide
gactctataggcggtacttacctatagtgagtcgtattaatttcg

SEQ ID NO: 18 - DNA, 45 bp, Artificial Sequence
  oligonucleotide
cgaaattaatacgactcactataggtaagtaccgcctatagagtc

SEQ ID NO: 19 - DNA, 43 bp, Artificial Sequence
  oligonucleotide
ccaacttttctggcggaattaatgcatctagggcggccaattc

SEQ ID NO: 20 - DNA, 53 bp, Artificial Sequence
  oligonucleotide
gtaaaacgacgggatctatcatggtggcggcggttggcaagcttatcatcgtg

SEQ ID NO: 21 - DNA, 53 bp, Artificial Sequence
  oligonucleotide
cacgatgataagcttgccaaccgccgccaccatgatagatcccgtcgttttac

SEQ ID NO: 22 - DNA, 32 bp, Artificial Sequence
  oligonucleotide
gcctcgagctatttttgacaccagaccaactg

SEQ ID NO: 23 - DNA, 46 bp, Artificial Sequence
  oligonucleotide
catttaggtgacactatagaatctatttttgacaccagaccaactg

SEQ ID NO: 24 - DNA, 46 bp, Artificial Sequence
  oligonucleotide
cagttggtctggtgtcaaaaatagattctatagtgtcacctaaatg

SEQ ID NO: 25 - DNA, 43 bp, Artificial Sequence
  oligonucleotide
gaattggccgccctagatgcattaattccgccagaaaagttgg

SEQ ID NO: 26 - DNA, 28 bp, Artificial Sequence
  oligonucleotide
ggcatcatgatagatcccgtcgttttac

SEQ ID NO: 27 - DNA, 20 bp, Artificial Sequence
  oligonucleotide
tactgtgagccagagttgcc

SEQ ID NO: 28 - DNA, 30 bp, Artificial Sequence
  oligonucleotide
gcgcggccgctctggttatccaggcttctg

SEQ ID NO: 29 - DNA, 41 bp, Artificial Sequence
  oligonucleotide
ggtggcggcggtaccaagcttgtgccaagagttccgacttg

SEQ ID NO: 30 - DNA, 41 bp, Artificial Sequence
  oligonucleotide
caagtcggaactcttggcacaagcttggtaccgccgccacc

SEQ ID NO: 31 - DNA, 19 bp, Artificial Sequence
  oligonucleotide
tgcttgcacctggaataag

SEQ ID NO: 32 - DNA, 19 bp, Artificial Sequence
  oligonucleotide
tgcttgcacctggaataag

SEQ ID NO: 33 - DNA, 22 bp, Artificial Sequence
  oligonucleotide
gagtctgcagtcctcgagaagc

SEQ ID NO: 34 - DNA, 22 bp, Artificial Sequence
  oligonucleotide
tgagtctgcagtcctcgagaag

SEQ ID NO: 35 - DNA, 25 bp, Artificial Sequence
  oligonucleotide
ctccaatcctccaaccagagccagc

SEQ ID NO: 36 - DNA, 25 bp, Artificial Sequence
  oligonucleotide
tccaatcctccaaccagagccagct

SEQ ID NO: 37 - DNA, 20 bp, Artificial Sequence
  oligonucleotide
aatctcgggtacgtgttccg

SEQ ID NO: 38 - DNA, 20 bp, Artificial Sequence
  oligonucleotide
ttggccaggtagatttctgc

SEQ ID NO: 39 - DNA, 20 bp, Artificial Sequence
  oligonucleotide
aatctcgggtacgtgttccg

SEQ ID NO: 40 - DNA, 20 bp, Artificial Sequence
  oligonucleotide
ttggccaggtagatttctgc

SEQ ID NO: 41 - DNA, 1707 bp, rat
gtagcctgcctccgatatttgttagaacaacggttccccgccacctaccaactgtttatgttttctctaacaaaaaccagaccggccgctgggcctgatacctgagttcagtcaccaagacccacgtggcagaaggagagaactgacttctgcatattatcctccaacacacacacacacacacacacacacacacacacacacacacacacacactaaaataaataaatagtctgggcttggtggcacattgagaacttacctcagaaaaaaggtaagtagataaagtaaaactaaaatggagtgagtcacactggagttccatgttaccaaattaaaactagctttctgaccttctgagaaaccaggacagaaagaggtgaaggccacattttctagccatgccaactgcagcaaacataactctgttctggctgccattgtccttatgaaaagtaagcaggagggatctgatctattaaccagctagctctgtgcttccctcctcttctcccaacctcccaaggaaaacatactccgtccttttcctttgttttattcctgcttcctgtctaggaaatcactcccctccaaggcgtcagaacacattctggcttacagaatgaagttttacccaattctagaatcacaaaatatagccaacgtaaaccttgaatgtgatctaattggtctaagaggcagaaatgagatgaagaaaaaaactgccgacatagatttcagtctatgggatgatgggcacataaacaataagaagaaagtgccagacaggggtaggtgctctaaatacaagataaattagagcaggttgagaagatggtactggggattggaggggcgactgctttaggcagggtatgggaaaggtatgccccctgagagaggatgttcatttttagcacttgaattttattttagtgtatgtgtatgcatgtgccacagcaaatgtatagaagtaaaaggagaccttgagagaagtggttcactcctcccatgttggtcttgggatcgaagtcaggttgttagacttgacaggaagtttctctccccagtgagctgtctcaccagcccaaagggtggcaacatttttgctgagacctaaataaaggacatgcgtcagttcagaaaccacagatatctgatcaaccaagctcctgcagtctcacctcatcttcctctcagccacactggcccttcagtggccccagcagtccccgaggtaggtggctcaaaatgtttatgtggctacctttcatcaactccttccccatctccagccccggccagaccctccagggcaaactgaggcctcatctgagctcctcctctgctcgcccaatccttccaaccccctatggtggtattgtctgtttaccctataggacatcctatagggtaaacagacaatagaccataggacaacaggcaggagcatgcctgctattgtcctcccttgtcctccctgccatcctaaagctggcaggtggctggtggtatatggaggatgtagctgggccagggaaaagatcctgcactaaaaatctgaagctaaaaataacaggacacgggatggaggagctcaggtggtatggctgacacagaaaatgtctgctcctgtatgggacatttgcccctcttctccaaatataagacaggatgaggcctagcttttgctgctccaaagtttta